(19) United States
(12) Patent Application Publication
(10) Pub. No.: US 2023/0352133 A1
(43) Pub. Date: Nov. 2, 2023

(54) SYSTEMS AND METHODS FOR PROCESSING MEDICAL DATA

(71) Applicant: Activ Surgical, Inc., Boston, MA (US)

(72) Inventors: Tina CHEN, Boston, MA (US); Roman STOLYAROV, Boston, MA (US); Thomas CALEF, Boston, MA (US); Tony CHEN, Boston, MA (US); Niall DALTON, Boston, MA (US); Jill BINNEY, Boston, MA (US); Vasiliy BUHARIN, Boston, MA (US); Bogdan MITREA, Boston, MA (US); Hossein DEHGHANI, Boston, MA (US); John OBERLIN, Boston, MA (US)

(21) Appl. No.: 18/061,922

(22) Filed: Dec. 5, 2022

Related U.S. Application Data
(63) Continuation of application No. PCT/US21/36236, filed on Jun. 7, 2021.
(60) Provisional application No. 63/036,293, filed on Jun. 8, 2020.
(60) Provisional application No. 63/166,842, filed on Mar. 26, 2021.

(51) Int. Cl. G16H 20/00; G16H 10/60; G16H 30/20; A61B 34/10

(52) U.S. Cl. CPC G16H 20/00; G16H 10/60; G16H 30/20; A61B 34/10; A61B 2034/105; A61B 2034/107

(57) ABSTRACT

The present disclosure provides methods for processing medical data. The method may comprise receiving a plurality of data inputs associated with (i) at least one medical patient or (ii) at least one surgical procedure. The method may further comprise receiving one or more annotations for at least a subset of the plurality of data inputs. The method may further comprise generating an annotated data set using (i) the one or more annotations and (ii) one or more data inputs of the plurality of data inputs. The method may further comprise using the annotated data set to (i) perform data analytics for the plurality of data inputs, (ii) develop one or more medical training tools, or (iii) train one or more medical models.

### CROSS-REFERENCE

This application is a continuation of International Patent Application No. PCT/US21/36236, filed Jun. 7, 2021, which claims priority to U.S. Provisional Patent Application No. 63/036,293 filed on Jun. 8, 2020, and U.S. Provisional Patent Application No. 63/166,842 filed on Mar. 26, 2021, each of which is incorporated herein by reference in its entirety for all purposes.

### BACKGROUND

Medical data for various patients and procedures may be compiled and analyzed to aid in the diagnosis and treatment of different medical conditions. Doctors and surgeons may utilize medical data compiled from various sources to make informed judgments about how to perform different medical operations. Medical data may be used by doctors and surgeons to perform complex medical procedures.

### SUMMARY

Surgeons may use annotated medical data to improve the detection and diagnosis of medical conditions, the treatment of medical conditions, and data analytics for live surgical procedures. Annotated medical data may also be provided to autonomous and semiautonomous robotic surgical systems to further enhance a surgeon's ability to detect, diagnose, and treat medical conditions. Systems and methods currently available for processing and analyzing medical data may be limited by the lack of large, clean datasets which are needed for surgeons to make accurate, nonbiased assessments. Processing and analyzing medical data may further require ground truth comparisons to verify the quality of data. The systems and methods disclosed herein may be used to generate accurate and useful datasets that can be leveraged for a variety of different medical applications. The systems and methods disclosed herein may be used to accumulate large datasets from reliable sources, verify the data provided from different sources, and improve the quality or value of aggregated data through crowdsourced annotations from medical experts and healthcare specialists. The systems and methods disclosed herein may be used to generate annotated datasets based on the current needs of a doctor or a surgeon performing a live surgical procedure, and to provide the annotated datasets to medical professionals or robotic surgical systems to enhance a performance of one or more surgical procedures. The annotated data sets generated using the systems and methods of the present disclosure may also improve the precision, flexibility, and control of robotic surgical systems. Surgical operators may benefit from autonomous and semiautonomous robotic surgical systems that can use the annotated data sets to augment information available to surgical operators during a surgical procedure. Such robotic surgical systems can further provide a medical operator with additional information through live updates or overlays to enhance a medical operator's ability to quickly and efficiently perform one or more steps of a live surgical procedure in an optimal manner.

In an aspect, the present disclosure provides systems and methods for data annotation.

In one aspect, a method for processing medical data is provided. The method comprises: (a) receiving a plurality of data inputs associated with (i) at least one medical patient or (ii) at least one surgical procedure; (b) receiving one or more annotations for at least a subset of the plurality of data inputs; (c) generating an annotated data set using (i) the one or more annotations and (ii) one or more data inputs of the plurality of data inputs; and (d) using the annotated data set to (i) perform data analytics for the plurality of data inputs, (ii) develop one or more medical training tools, or (iii) train one or more medical models.

In some embodiments, performing data analytics may comprise determining one or more factors that influence a surgical outcome. Performing data analytics may comprise generating statistics corresponding to one or more measurable characteristics associated with the plurality of data inputs or the one or more annotations. The statistics may correspond to a flow of a biological material in a perfusion map, a stitch tension during one or more steps of a stitching operation, tissue elasticity for one or more tissue regions, or a range of acceptable excision margins for a surgical procedure. Performing data analytics may comprise characterizing one or more surgical tasks associated with the at least one surgical procedure. The one or more medical training tools may be configured to provide best practices or guidelines for performing one or more surgical procedures. The one or more medical training tools may be configured to provide information on one or more optimal surgical tools for performing a surgical procedure. The one or more medical training tools may be configured to provide information on an optimal way to use a surgical tool. The one or more medical training tools may be configured to provide information on an optimal way to perform a surgical procedure. The one or more medical training tools may be configured to provide procedure training or medical instrument training. The one or more medical training tools may comprise a training simulator. The one or more medical training tools may be configured to provide outcome-based training for one or more surgical procedures.

In some embodiments, the above-described method may further comprise: (e) providing the one or more trained medical models to a controller that is in communication with one or more medical devices configured for autonomous or semi-autonomous surgery, wherein the controller is configured to implement the one or more trained medical models to aid one or more live surgical procedures. The at least one surgical procedure and the one or more live surgical procedures may be of a similar type of surgical procedure. Aiding the one or more live surgical procedures may comprise providing guidance to a surgeon while the surgeon is performing one or more steps of the one or more live surgical procedures. Aiding the one or more live surgical procedures may comprise improving a control or a motion of one or more robotic devices that are configured to perform autonomous or semi-autonomous surgery. Aiding the one or more live surgical procedures may comprise automating one or more surgical procedures.

In some embodiments, the plurality of data inputs may comprise medical data associated with the at least one medical patient. The medical data may comprise physiological data of the at least one medical patient. The physiological data may comprise an electrocardiogram, an electroencephalogram, an electromyogram, a blood pressure, a heart rate, a respiratory rate, or a body temperature of the at least one medical patient. The medical data may comprise medical imagery associated with the at least one medical patient. The medical imagery may comprise a pre-operative image selected from the group consisting of an ultrasound image, an X-ray image, an optical coherence tomography (OCT) scan, a computed tomography (CT) scan, a magnetic resonance imaging (MM) scan, and a positron emission tomography (PET) scan. The medical imagery may comprise an intraoperative image of a surgical scene or one or more streams of intraoperative data comprising the intraoperative image, wherein the intraoperative image may be selected from the group consisting of an RGB image, a depth map, a fluoroscopic image, a laser speckle contrast image, a hyperspectral image, a multispectral image, an ultrasound image, and a laser doppler image. The plurality of data inputs may comprise kinematic data associated with a movement of a robotic device or a medical instrument that is used to perform one or more steps of the at least one surgical procedure. The kinematic data may be obtained using an accelerometer or an inertial measurement unit. The plurality of data inputs may comprise kinetic data associated with a force, a stress, or a strain that is exerted on a tissue region of the at least one medical patient during the at least one surgical procedure. The plurality of data inputs may comprise an image or a video of the at least one surgical procedure. The plurality of data inputs may comprise an image or a video of one or more medical instruments used to perform the at least one surgical procedure. The plurality of data inputs may comprise instrument-specific data associated with (i) a physical characteristic of one or more medical instruments used to perform the at least one surgical procedure or (ii) a functional characteristic associated with an operation or a use of the one or more medical instruments during the at least one surgical procedure. The physical characteristic may comprise a geometry of the one or more medical instruments. The plurality of data inputs may comprise user control data corresponding to one or more inputs or motions by a medical operator to control a robotic device or a medical instrument to perform the at least one surgical procedure. The plurality of data inputs may comprise surgery-specific data associated with the at least one surgical procedure, wherein the surgery-specific data may comprise information on a type of surgery, a plurality of steps associated with the at least one surgical procedure, one or more timing parameters associated with the plurality of steps, or one or more medical instruments usable to perform the plurality of steps. The plurality of data inputs may comprise surgery-specific data associated with the at least one surgical procedure, wherein the surgery-specific data may comprise information on at least one of a relative position or a relative orientation of one or more ports through which a medical instrument or an imaging device is configured to be inserted. The plurality of data inputs may comprise patient-specific data associated with the at least one medical patient, wherein the patient-specific data may comprise one or more biological parameters of the at least one medical patient. The one or more biological parameters may correspond to a physical characteristic, a medical condition, or a pathological condition of the at least one medical patient. The patient-specific data may comprise anonymized or de-identified patient data. The plurality of data inputs may comprise robotic data associated with a movement of a robotic device to perform one or more steps of the at least one surgical procedure. The robotic device may comprise a robotic arm that is configured to move or control one or more medical instruments.

In some embodiments, the one or more medical models may be trained using neural networks or convolutional neural networks. The one or more medical models may be trained using one or more classical algorithms configured to implement exponential smoothing, single exponential smoothing, double exponential smoothing, triple exponential smoothing, Holt-Winters exponential smoothing, autoregressions, moving averages, autoregressive moving averages, autoregressive integrated moving averages, seasonal autoregressive integrated moving averages, vector autoregressions, or vector autoregression moving averages. The one or more medical models may be trained using deep learning. The deep learning may be supervised, unsupervised, or semi-supervised. The one or more medical models may be trained using reinforcement learning or transfer learning. The one or more medical models may be trained using image thresholding or color-based image segmentation. The one or more medical models may be trained using clustering. The one or more medical models may be trained using regression analysis. The one or more medical models may be trained using support vector machines. The one or more medical models may be trained using one or more decision trees or random forests associated with the one or more decision trees. The one or more medical models may be trained using dimensionality reduction. The one or more medical models may be trained using a recurrent neural network. The recurrent neural network may be a long short-term memory neural network. The one or more medical models may be trained using one or more temporal convolutional networks. The temporal convolutional networks may have a single or multiple stages. The one or more medical models may be trained using data augmentation techniques or generative adversarial networks.

In some embodiments, the one or more trained medical models may be configured to (i) receive a set of inputs corresponding to the one or more live surgical procedures or one or more surgical subjects of the one or more live surgical procedures and (ii) implement or perform one or more surgical applications, based at least in part on the set of inputs, to enhance a medical operator's ability to perform the one or more live surgical procedures. The set of inputs may comprise medical data associated with the one or more surgical subjects. The medical data may comprise physiological data of the one or more surgical subjects. The physiological data may comprise an electrocardiogram, electroencephalogram, an electromyogram, a blood pressure, a heart rate, a respiratory rate, or a body temperature of the one or more surgical subjects. The medical data may comprise medical imagery. The medical imagery may comprise a pre-operative image selected from the group consisting of an ultrasound image, an X-ray image, an optical coherence tomography (OCT) scan, a computed tomography (CT) scan, a magnetic resonance imaging (MRI) scan, and a positron emission tomography (PET) scan. The medical imagery may comprise an intraoperative image of a surgical scene or one or more streams of intraoperative data comprising the intraoperative image, wherein the intraoperative image may be selected from the group consisting of an RGB image, a depth map, a fluoroscopic image, a laser speckle contrast image, a hyperspectral image, a multispectral image, an ultrasound image, and a laser doppler image. The set of inputs may comprise kinematic data associated with a movement of a robotic device or a medical instrument that is usable to perform one or more steps of the one or more live surgical procedures. The kinematic data may be obtained using an accelerometer or an inertial measurement unit. The set of inputs may comprise kinetic data associated with a force, a stress, or a strain that is exerted on a tissue region of the one or more surgical subjects during the one or more live surgical procedures. The set of inputs may comprise an image or a video of the one or more live surgical procedures. The set of inputs may comprise an image or a video of one or more medical instruments used to perform the one or more live surgical procedures. The set of inputs may comprise instrument-specific data associated with (i) a physical characteristic of one or more medical instruments used to perform the one or more live surgical procedures or (ii) a functional characteristic associated with an operation or a use of the one or more medical instruments during the one or more live surgical procedures. The physical characteristic may comprise a geometry of the one or more medical instruments. The set of inputs may comprise user control data corresponding to one or more inputs or motions by the medical operator to control a medical instrument to perform the one or more live surgical procedures. The set of inputs may comprise surgery-specific data associated with the one or more live surgical procedures, wherein the surgery-specific data may comprise information on a type of surgery, a plurality of steps associated with the one or more live surgical procedures, one or more timing parameters associated with the plurality of steps, or one or more medical instruments usable to perform the plurality of steps. The set of inputs may comprise subject-specific data associated with the one or more surgical subjects, wherein the subject-specific data may comprise one or more biological parameters of the one or more surgical subjects. The one or more biological parameters may correspond to a physical characteristic, a medical condition, or a pathological condition of the one or more surgical subjects. The subject-specific data may comprise anonymized or de-identified subject data. The set of inputs may comprise robotic data associated with a movement or a control of a robotic device to perform one or more steps of the one or more live surgical procedures. The robotic device may comprise a robotic arm that is configured to move or control one or more medical instruments.

In some embodiments, the one or more surgical applications may comprise image segmentation. The image segmentation may be usable to identify one or more medical instruments used to perform the one or more live surgical procedures. The image segmentation may be usable to identify one or more tissue regions of the one or more surgical subjects undergoing the one or more live surgical procedures. The image segmentation may be usable to (i) distinguish between healthy and unhealthy tissue regions, or (ii) distinguish between arteries and veins. The one or more surgical applications may comprise object detection. The object detection may comprise detecting one or more deformable tissue regions or one or more rigid objects in a surgical scene. The one or more surgical applications may comprise scene stitching to stitch together two or more images of a surgical scene. The scene stitching may comprise generating a mini map corresponding to the surgical scene. The scene stitching may be implemented using an optical paintbrush. The one or more surgical applications may comprise sensor enhancement to augment one or more images or measurements obtained using one or more sensors with additional information associated with at least a subset of the set of inputs provided to the trained medical models. The sensor enhancement may comprise image enhancement. The image enhancement may comprise auto zoom into one or more portions of a surgical scene, auto focus on the one or more portions of a surgical scene, lens smudge removal, or an image correction. The one or more surgical applications may comprise generating one or more procedural inferences associated with the one or more live surgical procedures. The one or more procedural inferences may comprise an identification of one or more steps in a surgical procedure or a determination of one or more surgical outcomes associated with the one or more steps. The one or more surgical applications may comprise registering a pre-operative image of a tissue region of the one or more surgical subjects to one or more live images of the tissue region of the one or more surgical subjects obtained during the one or more live surgical procedures. The one or more surgical applications may comprise providing an augmented reality or virtual reality representation of a surgical scene. The augmented reality or virtual reality representation of the surgical scene may be configured to provide smart guidance for one or more camera operators to move one or more cameras relative to the surgical scene. The augmented reality or virtual reality representation of the surgical scene may be configured to provide one or more alternative camera or display views to a medical operator during the one or more live surgical procedures. The one or more surgical applications may comprise adjusting a position, an orientation, or a movement of one or more robotic devices or medical instruments during the one or more live surgical procedures. The one or more surgical applications may comprise coordinating a movement of two or more robotic devices or medical instruments during the one or more live surgical procedures. The one or more surgical applications may comprise coordinating a movement of a robotic camera and a robotically controlled medical instrument. The one or more surgical applications may comprise coordinating a movement of a robotic camera and a medical instrument that is manually controlled by the medical operator. The one or more surgical applications may comprise locating one or more landmarks in a surgical scene. The one or more surgical applications may comprise displaying physiological information associated with the one or more surgical subjects on one or more images of a surgical scene obtained during the one or more live surgical procedures. The one or more surgical applications may comprise safety monitoring, wherein safety monitoring may comprise geofencing one or more regions in a surgical scene or highlighting one or more regions in the surgical scene for the medical operator to target or avoid. The one or more surgical applications may comprise providing the medical operator with information on an optimal position, orientation, or movement of a medical instrument to perform one or more steps of the one or more live surgical procedures. The one or more surgical applications may comprise informing the medical operator of one or more surgical instruments or surgical methods for performing one or more steps of the one or more live surgical procedures. The one or more surgical applications may comprise informing the medical operator of an optimal stitch pattern. The one or more surgical applications may comprise measuring perfusion, stitch tension, tissue elasticity, or excision margins. The one or more surgical applications may comprise measuring a distance between a first tool and a second tool in real time. The distance between the first tool and the second tool may be measured based at least in part on a geometry of the first tool and the second tool. The distance between the first tool and the second tool may be measured based at least in part on a relative position or a relative orientation of a scope that is used to perform the one or more live surgical procedures. The method may further comprise detecting one or more edges of the first tool or the second tool to determine a position and an orientation of the first tool relative to the second tool. The method may further comprise determining a three-dimensional position of a tool tip of the first tool and a three-dimensional position of a tool tip of the second tool. The method may further comprise registering a scope port to a pre-operative image to determine a position and an orientation of the first tool, the second tool, and the scope relative to one or more tissue regions of a surgical patient. The one or more surgical applications may comprise measuring a distance between a tool and a scope in real time. The distance between the tool and the scope may be measured based at least in part on a geometry of the first tool and the scope. The distance between the tool and the scope may be measured based at least in part on a relative position or a relative orientation of the scope. The method may further comprise detecting one or more edges of the tool or the scope to determine a position and an orientation of the tool relative to the scope. The method may further comprise using the one or more detected edges of the tool or the scope to improve position feedback of the tool or the scope. The method may further comprise detecting a global position or a global orientation of the scope using an inertial measurement unit. The method may further comprise detecting a global position or a global orientation of one or more tools within a surgical scene based at least in part on (i) the global position or global orientation of the scope and (ii) a relative position or a relative orientation of the one or more tools in relation to the scope. The method may further comprise determining a depth of camera insertion based at least in part on (i) the global position or the global orientation of the scope, (ii) the global position or the global orientation of the one or more tools, or (iii) the relative position or the relative orientation of the one or more tools in relation to the scope. The method may further comprise determining a depth of tool insertion based at least in part on (i) the global position or the global orientation of the scope, (ii) the global position or the global orientation of the one or more tools, or (iii) the relative position or the relative orientation of the one or more tools in relation to the scope. The method may further comprise predicting an imaging region of a camera based at least in part on an estimated or a priori knowledge of (i) a position or an orientation of the camera or (ii) a position or an orientation of a scope port through which the camera is inserted. The method may further comprise determining a three-dimensional position of a tool tip of the tool and a three-dimensional position of a tip of the scope. The method may further comprise registering a scope port to a pre-operative image to determine a position and an orientation of the tool and the scope relative to one or more tissue regions of a surgical patient. The one or more surgical applications may comprise displaying one or more virtual representations of one or more tools in a pre-operative image of a surgical scene. The one or more surgical applications may comprise displaying one or more virtual representations of one or more medical instruments in a live image or video of a surgical scene. The one or more surgical applications may comprise determining one or more dimensions of a medical instrument. The one or more surgical applications may comprise determining one or more dimensions of a critical structure of the one or more surgical subjects. The one or more surgical applications may comprise providing an overlay of a perfusion map and a pre-operative image of a surgical scene. The one or more surgical applications may comprise providing an overlay of a perfusion map and a live image of a surgical scene. The one or more surgical applications may comprise providing an overlay of a pre-operative image of a surgical scene and a live image of the surgical scene. The one or more surgical applications may comprise providing a set of virtual markers to guide the medical operator during one or more steps of the one or more live surgical procedures.

In some embodiments, the one or more annotations may comprise a bounding box that is generated around one or more portions of the medical imagery. The one or more annotations may comprise a zero-dimensional feature that is generated within the medical imagery. The zero-dimensional feature may comprise a dot. The one or more annotations may comprise a one-dimensional feature that is generated within the medical imagery. The one-dimensional feature may comprise a line, a line segment, or a broken line comprising two or more line segments. The one-dimensional feature may comprise a linear portion. The one-dimensional feature may comprise a curved portion. The one or more annotations may comprise a two-dimensional feature that is generated within the medical imagery. The two-dimensional feature may comprise a circle, an ellipse, or a polygon with three or more sides. The two-dimensional feature may comprise a shape with two or more sides having different lengths or different curvatures. The two-dimensional feature may comprise a shape with one or more linear portions. The two-dimensional feature may comprise a shape with one or more curved portions. The two-dimensional feature may comprise an amorphous shape that does not correspond to a circle, an ellipse, or a polygon. The one or more annotations may comprise a textual annotation to the medical data associated with the at least one medical patient. The one or more annotations may comprise a textual, numerical, or visual indication of an optimal position, orientation, or movement of the robotic device or the medical instrument. The one or more annotations may comprise one or more labeled windows or timepoints to a data signal corresponding to the movement of the robotic device or the medical instrument. The one or more annotations may comprise a textual, numerical, or visual suggestion on how to move the robotic device or the medical instrument to optimize performance of the one or more steps of the at least one surgical procedure. The one or more annotations may comprise an indication of when the robotic device or the medical instrument is expected to enter a field of view of an imaging device that is configured to monitor a surgical scene associated with the at least one surgical procedure. The one or more annotations may comprise an indication of an estimated position or an estimated orientation of the robotic device or the medical instrument during the one or more steps of the at least one surgical procedure. The one or more annotations may comprise an indication of an estimated direction in which the robotic device or the medical instrument is moving relative to a surgical scene associated with the at least one surgical procedure during the one or more steps of the at least one surgical procedure. The one or more annotations may comprise one or more markings that may be configured to indicate an optimal position or an optimal orientation of a camera to visualize the one or more steps of the at least one surgical procedure at a plurality of time instances. The one or more annotations may comprise a textual, numerical, or visual indication of an optimal stress, strain, or force on a tissue region during a surgical procedure. The one or more annotations may comprise a textual, numerical, or visual indication of an optimal stress, strain, or force on a tissue region during a suturing procedure. The one or more annotations may comprise a textual, numerical, or visual indication of an optimal angle or an optimal direction of motion of a needle relative to a tissue region during a suturing procedure. The one or more annotations may comprise a visual indication of an optimal stitching pattern. The one or more annotations may comprise a visual marking on the image or the video of the at least one surgical procedure. The one or more annotations may comprise a visual marking on the image or the video of the one or more medical instruments used to perform the at least one surgical procedure. The one or more annotations may comprise one or more textual, numerical, or visual annotations to the user control data to indicate an optimal input or an optimal motion by the medical operator to control the robotic device or the medical instrument. The one or more annotations may comprise one or more textual, numerical, or visual annotations to the robotic data to indicate an optimal movement of the robotic device to perform the one or more steps of the at least one surgical procedure.

In some embodiments, the method may further comprise validating the plurality of data inputs prior to receiving the one or more annotations. Validating the plurality of data inputs may comprise scoring the plurality of data inputs, retaining at least a first subset of the plurality of data inputs with a first set of scores that is above a pre-determined threshold, and discarding at least a second subset of the plurality of data inputs with a second set of scores that is below the pre-determined threshold. The method may further comprise validating the one or more annotations prior to training the medical models. Validating the one or more annotations may comprise scoring the one or more annotations, retaining at least a first subset of the one or more annotations with a first set of scores that is above a pre-determined threshold, and discarding at least a second subset of the one or more annotations with a second set of scores that is below the pre-determined threshold. The method may further comprise grading one or more annotators who provided or generated the one or more annotations. Grading the one or more annotators may comprise ranking the one or more annotators based on a level of expertise of the one or more annotators or a level of quality associated with the one or more annotations provided by the one or more annotators. Grading the one or more annotators may comprise assigning a level of expertise to the one or more annotators based on a level of quality associated with the one or more annotations provided by the one or more annotators. The one or more annotations may be aggregated using crowd sourcing. The plurality of data inputs may be aggregated using crowd sourcing. The plurality of data inputs may be provided to a cloud server for annotation. The one or more annotations may be generated or provided by one or more annotators using a cloud-based platform. The one or more annotations may be stored on a cloud server.

In another aspect, the present disclosure provides a method for generating medical insights, comprising: (a) obtaining medical data associated with a surgical procedure using one or more medical tools or instruments; (b) processing the medical data using one or more medical algorithms or models, wherein the one or more medical algorithms or models are deployed or implemented on or by (i) the one or more medical tools or instruments or (ii) a data processing platform; (c) generating one or more insights or inferences based on the processed medical data; and (d) providing the one or more insights or inferences for the surgical procedure to at least one of (i) a device in an operating room and (ii) a user via the data processing platform.

In some embodiments, the method further comprises registering the one or more medical tools or instruments with the data processing platform. In some embodiments, the method further comprises uploading the medical data or the processed medical data from the one or more medical tools or instruments to the data processing platform. In some embodiments, the one or more medical algorithms or models are trained using one or more data annotations provided for one or more medical data sets. In some embodiments, the one or more medical data sets are associated with one or more reference surgical procedures of a same or similar type as the surgical procedure. In some embodiments, the one or more medical tools or instruments comprise an imaging device. In some embodiments, the imaging device is configured for RGB imaging, laser speckle imaging, fluorescence imaging, or time of flight imaging. In some embodiments, the medical data comprises one or more images or videos of the surgical procedure or one or more steps of the surgical procedure. In some embodiments, processing the medical data comprises determining or classifying one or more features, patterns, or attributes of the medical data. In some embodiments, the one or more insights comprise tool identification, tool tracking, surgical phase timeline, critical view detection, tissue structure segmentation, and/or feature detection. In some embodiments, the one or more medical algorithms or models are configured to perform tissue tracking. In some embodiments, the one or more medical algorithms or models are configured to augment the medical data with depth information. In some embodiments, the one or more medical algorithms or models are configured to perform tool segmentation, phase of surgery breakdown, critical view detection, tissue structure segmentation, and/or feature detection. In some embodiments, the one or more medical algorithms or models are configured to perform deidentification or anonymization of the medical data. In some embodiments, the one or more medical algorithms or models are configured to provide live guidance based on a detection of one or more tools, surgical phases, critical views, or one or more biological, anatomical, physiological, or morphological features in or near the surgical scene. In some embodiments, the one or more medical algorithms or models are configured to generate synthetic data for simulation and/or extrapolation. In some embodiments, the one or more medical algorithms or models are configured to assess a quality of the medical data. In some embodiments, the one or more medical algorithms or models are configured to generate an overlay comprising (i) one or more RGB images or videos of the surgical scene and (ii) one or more additional images or videos of the surgical procedure, wherein the one or more additional images or videos comprise fluorescence data, laser speckle data, perfusion data, or depth information. In some embodiments, the one or more medical algorithms or models are configured to provide one or more surgical inferences. In some embodiments, the one or more inferences comprise a determination of whether a tissue is alive. In some embodiments, the one or more inferences comprise a determination of where to make a cut or an incision. In some embodiments, the one or more medical algorithms or models are configured to provide virtual surgical assistance to a surgeon or a doctor performing the surgical procedure.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically illustrates a flow diagram for processing medical data, in accordance with some embodiments.

FIG. 1B schematically illustrates a platform for processing medical data, in accordance with some embodiments.

FIG. 1C schematically illustrates a user interface of the platform for processing medical data, in accordance with some embodiments.

FIG. 1D schematically illustrates an example of surgical insights comprising a timeline of a surgical procedure, in accordance with some embodiments.

FIG. 1E schematically illustrates an example of surgical insights comprising augmented visualizations of a surgical scene, in accordance with some embodiments.

FIG. 1F schematically illustrates an example of surgical insights comprising tool segmentation, in accordance with some embodiments.

FIG. 1G schematically illustrates a user interface for manually uploading surgical data or surgical videos, in accordance with some embodiments.

FIG. 2 schematically illustrates a flow diagram for annotating medical data, in accordance with some embodiments.

FIG. 3 schematically illustrates an exemplary method for processing medical data, in accordance with some embodiments.

FIG. 4A schematically illustrates a surgical video of a surgical scene, in accordance with some embodiments.

FIG. 4B schematically illustrates a detection of tool edges within a surgical video, in accordance with some embodiments.

FIG. 5A schematically illustrates a visual representation of a position and an orientation of a scope relative to a surgical scene, in accordance with some embodiments.

FIG. 5B schematically illustrates a visual representation of a position and an orientation of one or more surgical tools relative to a scope, in accordance with some embodiments.

FIG. 6A schematically illustrates a plurality of tool tips detected within a surgical video, in accordance with some embodiments.

FIG. 6B schematically illustrates a visual representation of an estimated three-dimensional (3D) position of one or more tool tips relative to a scope, in accordance with some embodiments.

FIG. 7 schematically illustrates an augmented reality view of a surgical scene showing a tip-to-tip distance between one or more medical tools and tip-to-scope distances between a scope and one or more medical tools, in accordance with some embodiments.

FIGS. 8A and 8B schematically illustrate one or more virtual views of one or more medical tools inside a patient, in accordance with some embodiments.

FIG. 9A schematically illustrates a surgical video of a tissue region of a patient, in accordance with some embodiments.

FIG. 9B schematically illustrates a visualization of RGB and perfusion data associated with a tissue region of the patient, in accordance with some embodiments.

FIG. 10A schematically illustrates a surgical video of a tissue region of a medical patient or surgical subject, in accordance with some embodiments.

FIG. 10B schematically illustrates annotated data that may be generated for a surgical video of a tissue region of a surgical subject, in accordance with some embodiments.

FIG. 10C schematically illustrates a real-time display of augmented visuals and surgical guidance indicating where to make a cut, in accordance with some embodiments.

FIG. 11 schematically illustrates a computer system that is programmed or otherwise configured to implement methods provided herein.

FIG. 12 schematically illustrates a critical view of safety during a surgical procedure, in accordance with some embodiments.

FIG. 13 schematically illustrates a machine learning development pipeline, in accordance with some embodiments.

FIG. 14 schematically illustrates an example of an annotated and augmented medical image or video frame, in accordance with some embodiments.

FIG. 15 schematically illustrates an example of a perfusion overlay, in accordance with some embodiments.

FIG. 16 schematically illustrates converting a model from one or more training frameworks to an open standard, in accordance with some embodiments.

FIG. 17 schematically illustrates inference latencies for various Open Neural Network Exchange (ONNX) runtime execution providers, in accordance with some embodiments.

FIG. 18 schematically illustrates a pipeline for creating a TensorRT engine, in accordance with some embodiments.

FIG. 19 schematically illustrates a comparison of latencies of variants of a convolutional neural network across different devices, in accordance with some embodiments.

FIG. 20 schematically illustrates an example of a model training pipeline, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In an aspect, the present disclosure provides systems and methods for processing medical data. The systems and methods disclosed herein may be used to generate accurate and useful datasets that can be leveraged for a variety of different medical applications. The systems and methods disclosed herein may be used to accumulate large datasets from reliable sources, verify the data provided from different sources, and improve the quality or value of aggregated data through crowdsourced annotations from medical experts and healthcare specialists. The systems and methods disclosed herein may be used to generate annotated datasets based on the current needs of a doctor or a surgeon performing a live surgical procedure, and to provide the annotated datasets to medical professionals or robotic surgical systems to enhance a performance of one or more surgical procedures. The annotated data sets generated using the systems and methods of the present disclosure may also improve the precision, flexibility, and control of robotic surgical systems. Surgical operators may benefit from autonomous and semiautonomous robotic surgical systems that can use the annotated data sets to augment information available to surgical operators during a surgical procedure. Such robotic surgical systems can further provide a medical operator with additional information through live updates or overlays to enhance a medical operator's ability to quickly and efficiently perform one or more steps of a live surgical procedure in an optimal manner.

In an aspect, the present disclosure provides a method for processing medical data. The method may comprise (a) receiving a plurality of data inputs associated with (i) at least one medical patient or (ii) at least one surgical procedure. The method may further comprise (b) receiving one or more annotations for at least a subset of the plurality of data inputs. The method may further comprise (c) generating an annotated data set using (i) the one or more annotations and (ii) one or more data inputs of the plurality of data inputs. The method may further comprise (d) using the annotated data set to (i) perform data analytics for the plurality of data inputs, (ii) develop one or more medical training tools, or (iii) train one or more medical models.

In some cases, the method may further comprise (e) providing the one or more trained medical models to a controller that is in communication with one or more medical devices. In some cases, the one or more medical devices may be configured for autonomous or semi-autonomous surgery. In some cases, the controller may be configured to implement the one or more trained medical models to aid one or more live surgical procedures.

Data Inputs

The method may comprise (a) receiving a plurality of data inputs associated with (i) at least one medical patient or (ii) at least one surgical procedure. The plurality of data inputs may be obtained from one or more data providers. The one or more data providers may comprise one or more doctors, surgeons, medical professionals, medical facilities, medical institutions, and/or medical device companies. In some cases, the plurality of data inputs may be obtained using one or more medical devices and/or one or more medical imaging devices. The plurality of data inputs may be aggregated using one or more aspects of crowd sourcing. The plurality of data inputs may be provided to a cloud server for processing (e.g., ranking, quality control, validation, annotation, etc.).

The plurality of data inputs may be associated with at least one medical patient. The at least one medical patient may be a human. The at least one medical patient may be an individual who is undergoing, has undergone, or will be undergoing at least one surgical procedure.

The plurality of data inputs may be associated with at least one surgical procedure. The at least one surgical procedure may comprise one or more surgical procedures that are performed or performable using one or more medical tools or instruments. In some cases, the medical tools or instruments may comprise an endoscope or a laparoscope. In some cases, the one or more surgical procedures may be performed or performable using one or more robotic devices. The one or more robotic devices may be autonomous and/or semi-autonomous.

In some cases, the at least one surgical procedure may comprise one or more general surgical procedures, neurosurgical procedures, orthopedic procedures, and/or spinal procedures. In some cases, the one or more surgical procedures may comprise colectomy, cholecystectomy, appendectomy, hysterectomy, thyroidectomy, and/or gastrectomy. In some cases, the one or more surgical procedures may comprise hernia repair, and/or one or more suturing operations. In some cases, the one or more surgical procedures may comprise bariatric surgery, large or small intestine surgery, colon surgery, hemorrhoid surgery, and/or biopsy (e.g., liver biopsy, breast biopsy, tumor or cancer biopsy, etc.).

In some cases, the at least one surgical procedure associated with the plurality of data inputs may be of a same or similar type of surgical procedure as one or more live surgical procedures being performed with aid of one or more medical models that are generated and/or trained using the plurality of data inputs and one or more annotations for at least a subset of the data inputs.

Physiological Data/Medical Imagery

The plurality of data inputs may comprise medical data associated with the at least one medical patient. In some cases, the medical data may comprise physiological data of the at least one medical patient. The physiological data may comprise an electrocardiogram (ECG or EKG), an electroencephalogram (EEG), an electromyogram (EMG), a blood pressure, a heart rate, a respiratory rate, or a body temperature of the at least one medical patient.

The plurality of data inputs may comprise patient-specific data associated with the at least one medical patient. In some cases, the patient-specific data may comprise one or more biological parameters of the at least one medical patient. The one or more biological parameters may correspond to a physical characteristic, a medical condition, or a pathological condition of the at least one medical patient. In some cases, the patient-specific data may comprise anonymized or de-identified patient data.

The plurality of data inputs may comprise medical imagery associated with the at least one medical patient. In some cases, the medical imagery may comprise a pre-operative image selected from the group consisting of an ultrasound image, an X-ray image, an optical coherence tomography (OCT) scan, a computed tomography (CT) scan, a magnetic resonance imaging (MM) scan, and a positron emission tomography (PET) scan.

In some cases, the medical imagery may comprise an intraoperative image of a surgical scene. The intraoperative image may comprise an RGB image, a depth map, a fluoroscopic image, a laser speckle contrast image, a hyperspectral image, a multispectral image, an ultrasound image, and/or a laser doppler image. In some cases, the medical imagery may comprise one or more streams of intraoperative data comprising the intraoperative image. The one or more streams of intraoperative data may comprise a series of intraoperative images obtained successively or sequentially over a time period.

In some cases, the plurality of data inputs may comprise one or more images and/or one or more videos of the at least one surgical procedure. In some cases, the plurality of data inputs may comprise one or more images and/or one or more videos of one or more medical instruments used to perform the at least one surgical procedure.

Kinematic Data

The plurality of data inputs may comprise kinematic data associated with a movement of a robotic device or a medical instrument that is used to perform one or more steps of the at least one surgical procedure. In some cases, the kinematic data is obtained using an accelerometer or an inertial measurement unit. The kinematic data may comprise a position, a velocity, an acceleration, an orientation, and/or a pose of the robotic device, a portion of the robotic device, a medical instrument, and/or a portion of the medical instrument.

In some cases, the plurality of data inputs comprise user control data corresponding to one or more inputs or motions by a medical operator to control a robotic device or a medical instrument to perform the at least one surgical procedure. In some cases, the one or more inputs or motions by a medical operator to control a robotic device or a medical instrument may be associated with the kinematic data corresponding to an operation or a movement of the robotic device or the medical instrument.

In some cases, the plurality of data inputs may comprise robotic data associated with a movement of a robotic device to perform one or more steps of the at least one surgical procedure. In some cases, the robotic device may comprise a robotic arm that is configured to move or control one or more medical instruments.

Kinetic Data

The plurality of data inputs may comprise kinetic data associated with a force, a stress, or a strain that is exerted on a tissue region of the at least one medical patient during the at least one surgical procedure. The kinetic data may be associated with a movement of a robotic device or a robotic arm. In some cases, the kinetic data may be associated with a movement of a medical instrument that is coupled to the robotic device or the robotic arm.

Instrument Data

The plurality of data inputs may comprise instrument-specific data associated with (i) a physical characteristic of one or more medical instruments used to perform the at least one surgical procedure or (ii) a functional characteristic associated with an operation or a use of the one or more medical instruments during the at least one surgical procedure. In some cases, the physical characteristic may comprise a shape, a geometry, or a dimension (e.g., length, width, depth, height, thickness, diameter, circumference, etc.) of the one or more medical instruments. In some cases, the functional characteristic may comprise a mode of operation, a speed, a power, an intensity, a temperature, a frequency, a wavelength, a level of accuracy, and/or a level of precision associated with the one or more medical instruments.

Surgery Data

The plurality of data inputs may comprise surgery-specific data associated with the at least one surgical procedure. In some cases, the surgery-specific data may comprise information on a type of surgery, a plurality of steps associated with the at least one surgical procedure, one or more timing parameters associated with the plurality of steps (e.g., estimated time to complete the plurality of steps, estimated time to perform one or more steps, actual time needed to complete the plurality of steps, and/or actual time needed to perform one or more steps), or one or more medical instruments usable to perform the plurality of steps. In some cases, the surgery-specific data may comprise information on at least one of a relative position or a relative orientation of one or more ports through which a medical instrument or an imaging device may be inserted. The one or more ports may correspond to a portion of a trocar through which the medical instrument or the imaging device may be inserted. In some cases, the one or more ports may correspond to an incision on a portion of a subject's body. In some cases, the incision may be a keyhole incision.

Awarding Data Providers

In some cases, one or more surgical data sets may be requested from the one or more data providers. The one or more surgical data sets may comprise any of the data inputs described herein. In some cases, the one or more data providers may be awarded for supplying different types of data inputs or different metadata (e.g., procedure type or equipment used) associated with the different types of data. In some cases, a dynamic award system may be used in combination with the systems and methods disclosed herein. The dynamic award system may be configured to award data providers based on a need for or a lack of certain types of data or metadata. In some cases, the dynamic award system may be configured to award data providers based on a level of quality of the data inputs generated and/or provided by the data providers.

Ranking Data Inputs/Quality Assurance

In some cases, the plurality of data inputs may undergo quality assurance to evaluate and/or verify a level of quality associated with the data inputs. In some embodiments, the method may further comprise validating the plurality of data inputs prior to receiving the one or more annotations. Validating the plurality of data inputs may comprise scoring the plurality of data inputs, retaining at least a first subset of the plurality of data inputs with a first set of scores that is above a pre-determined threshold, and discarding at least a second subset of the plurality of data inputs with a second set of scores that is below the pre-determined threshold.

Ranking Data Providers

In some cases, the method may further comprise grading one or more data providers who provided or generated the plurality of data inputs. Grading the one or more data providers may comprise ranking the one or more data providers based on a level of expertise of the one or more data providers or a level of quality associated with the plurality of data inputs provided by the one or more data providers. Grading the one or more data providers may comprise assigning a level of expertise to the one or more data providers based on a level of quality associated with the plurality of data inputs provided by the one or more data providers.

Annotations

The method may further comprise (b) receiving one or more annotations for at least a subset of the plurality of data inputs. The method may further comprise (c) generating an annotated data set using (i) the one or more annotations and (ii) one or more data inputs from the plurality of data inputs.

The plurality of data inputs may be provided to and/or stored on a data annotation platform. The data annotation platform may comprise a cloud server. The data annotation platform may be configured to enable one or more annotators to access the plurality of data inputs and to provide one or more annotations for at least a subset of the plurality of data inputs. The one or more annotations may be aggregated using crowd sourcing. The data annotation platform may comprise a server that is accessible by one or more annotators via a communications network. The server may comprise a cloud server.

The one or more annotators may comprise one or more doctors, surgeons, nurses, medical professionals, medical institutions, medical students, medical residents, medical interns, medical staff, and/or medical researchers. In some cases, the one or more annotators may comprise one or more medical experts in a medical specialty. In some cases, the one or more annotators may comprise one or more data providers as described elsewhere herein. In some cases, the one or more annotators may comprise individuals or entities who do not have a medical background. In such cases, the one or more annotations provided by such individuals or entities who do not have medical backgrounds may be verified by one or more annotators with medical knowledge, experience, or expertise, for quality assurance purposes.

The one or more annotators may provide one or more annotations to at least a subset of the plurality of data inputs. The one or more annotations may be generated or provided by the one or more annotators using a cloud-based platform. The one or more annotations may be stored on a cloud server. The one or more annotations provided by the one or more annotators may be used to generate an annotated data set from the plurality of data inputs. The annotated data set may comprise one or more annotated data inputs.

Types of Annotations

In some cases, the one or more annotations may comprise a bounding box that is generated around one or more portions of the medical imagery. In some cases, the one or more annotations may comprise a zero-dimensional feature that is generated within the medical imagery. The zero-dimensional feature may comprise a dot. In some cases, the one or more annotations may comprise a one-dimensional feature that is generated within the medical imagery. The one-dimensional feature may comprise a line, a line segment, or a broken line comprising two or more line segments. In some cases, the one-dimensional feature may comprise a linear portion. In some cases, the one-dimensional feature may comprise a curved portion. In some cases, the one or more annotations may comprise a two-dimensional feature that is generated within the medical imagery. In some cases, the two-dimensional feature may comprise a circle, an ellipse, or a polygon with three or more sides. In some cases, two or more sides of the polygon may comprise a same length. In other cases, two or more sides of the polygon may comprise different lengths. In some cases, the two-dimensional feature may comprise a shape with two or more sides having different lengths or different curvatures. In some cases, the two-dimensional feature may comprise a shape with one or more linear portions and/or one or more curved portions. In some cases, the two-dimensional feature may comprise an amorphous shape that does not correspond to a circle, an ellipse, or a polygon. In some cases, the two-dimensional feature may comprise an arbitrary segmentation shape that is drawn or generated by an annotator.

In some cases, the one or more annotations may comprise a textual annotation to the medical data associated with the at least one medical patient. In some cases, the one or more annotations may comprise a textual, numerical, or visual indication of an optimal position, orientation, or movement of the robotic device or the medical instrument. In some cases, the one or more annotations may comprise one or more labeled windows or timepoints to a data signal corresponding to the movement of the robotic device or the medical instrument. In some cases, the labeled windows or timepoints may be used for data signals other than robotic movements and medical instruments. For example, the labeled windows or timepoints may be used to label the steps of a live, ongoing surgical procedure. Further, the labeled windows or timepoints may be used to indicate when fluorescence and/or other imaging modalities are being used (e.g., infrared, magnetic resonance imaging, X-ray, ultrasound, medical radiation, angiography, computed tomography, positron emission tomography, etc). In some cases, the labeled windows or timepoints may be used to indicate when a critical view of safety is achieved. In some cases, the one or more annotations may comprise a textual, numerical, or visual suggestion on how to move the robotic device or the medical instrument to optimize performance of the one or more steps of the at least one surgical procedure. In some cases, the one or more annotations may comprise an indication of when the robotic device or the medical instrument is expected to enter a field of view of an imaging device that is configured to monitor a surgical scene associated with the at least one surgical procedure. The imaging device may comprise a camera. In some cases, the one or more annotations may comprise an indication of an estimated position or an estimated orientation of the robotic device or the medical instrument during the one or more steps of the at least one surgical procedure. In some cases, the one or more annotations may comprise an indication of an estimated direction in which the robotic device or the medical instrument is moving relative to a surgical scene associated with the at least one surgical procedure during the one or more steps of the at least one surgical procedure. In some cases, the one or more annotations may comprise one or more markings that are configured to indicate an optimal position or an optimal orientation of a camera to visualize the one or more steps of the at least one surgical procedure at a plurality of different time instances.

In some cases, the one or more annotations may comprise a textual, numerical, or visual indication of an optimal stress, strain, or force on a tissue region during a surgical procedure. In some cases, the one or more annotations may comprise a textual, numerical, or visual indication of an optimal stress, strain, or force on a tissue region during a suturing procedure. In some cases, the one or more annotations may comprise a textual, numerical, or visual indication of an optimal angle or an optimal direction of motion of a needle relative to a tissue region during a suturing procedure. In some cases, the one or more annotations may comprise a visual indication of an optimal stitching pattern.

In some cases, the one or more annotations may comprise a visual marking on the image or the video of the at least one surgical procedure. In some cases, the one or more annotations may comprise a visual marking on the image or the video of the one or more medical instruments used to perform the at least one surgical procedure.

In some cases, the one or more annotations may comprise one or more textual, numerical, or visual annotations to the user control data to indicate an optimal input or an optimal motion by the medical operator to control the robotic device or the medical instrument. In some cases, the one or more annotations may comprise one or more textual, numerical, or visual annotations to the robotic data to indicate an optimal movement of the robotic device to perform the one or more steps of the at least one surgical procedure.

Ranking Annotations/Quality Assurance

The one or more annotations may be graded and/or ranked to indicate a quality or an accuracy of the one or more annotations. In some cases, the method may further comprise validating the one or more annotations prior to training the medical models. Validating the one or more annotations may comprise scoring the one or more annotations, retaining at least a first subset of the one or more annotations with a first set of scores that is above a pre-determined threshold, and discarding at least a second subset of the one or more annotations with a second set of scores that is below the pre-determined threshold.

Ranking/Rewarding Annotators

In some cases, the method may further comprise grading one or more annotators who provided or generated the one or more annotations. Grading the one or more annotators may comprise ranking the one or more annotators based on a level of expertise of the one or more annotators or a level of quality associated with the one or more annotations provided by the one or more annotators. Grading the one or more annotators may comprise assigning a level of expertise to the one or more annotators based on a level of quality associated with the one or more annotations provided by the one or more annotators. Different levels of expertise may be designated or required for different annotations required for certain data sets. In such cases, data annotators may be awarded or compensated based on a dynamic scale that adjusts depending on the level of expertise required to generate one or more data annotations tasks with a desired level of quality, precision, and/or accuracy. In some cases, data annotators may be awarded or compensated based on a level of quality of the annotations provided by the data annotators.

Synchronization

In some cases, the plurality of data inputs may comprise two or more data inputs of a same type. In other cases, the plurality of data inputs may comprise two or more data inputs of different types. In any of the embodiments described herein, the plurality of data inputs may be synchronized. Synchronization of the plurality of data inputs may comprise one or more spatial synchronization, one or more temporal synchronization, and/or one or more synchronizations with respect to a type of patient or a type of surgical procedure.

Data Analytics

In some cases, the method may comprise (d) using the annotated data set to (i) perform data analytics for the plurality of data inputs. Performing data analytics may comprise determining, from the plurality of data inputs and/or the one or more annotations, one or more factors associated with a medical patient and/or a surgical procedure that can influence a surgical outcome. In some cases, performing data analytics may comprise generating statistics corresponding to one or more measurable characteristics associated with the plurality of data inputs and/or the one or more annotations to the plurality of data inputs. In some cases, performing data analytics may comprise generating statistics corresponding to a flow of a biological material in a perfusion map, a stitch tension during a surgical procedure, a tissue elasticity for one or more tissue regions, or a range of acceptable excision margins for a surgical procedure. In some cases, performing data analytics may comprise characterizing one or more surgical tasks associated with the at least one surgical procedure. Characterizing one or more surgical tasks may comprise identifying one or more steps in a surgical procedure, identifying one or more optimal tools for performing or completing the one or more steps, identifying one or more optimal surgical techniques to perform or complete the one or more steps, or determining one or more timing parameters associated with the one or more steps. The one or more timing parameters may comprise an estimated or actual amount of time needed to complete the one or more steps.

Medical Training

In some cases, the method may comprise (d) using the annotated data set to (ii) develop one or more medical training tools. The one or more medical training tools may be used and/or deployed to train one or more doctors, surgeons, nurses, medical assistants, medical staff, medical workers, medical students, medical residents, medical interns, or healthcare providers. The one or more medical training tools may be configured to provide best practices or guidelines for performing one or more surgical procedures. The one or more medical training tools may be configured to provide information on one or more optimal surgical tools for performing a surgical procedure. The one or more medical training tools may be configured to provide information on an optimal way to use a surgical tool. The one or more medical training tools may be configured to provide information on an optimal way to perform a surgical procedure. The one or more medical training tools may be configured to provide procedure training or medical instrument training. The one or more medical training tools may be configured to provide outcome-based training for one or more surgical procedures. In some cases, the one or more medical training tools may comprise a training simulator. The training simulator may be configured to provide a trainee with a visual and/or virtual representation of a surgical procedure.

Training Methods for Medical Models

In some cases, the method may further comprise (d) using the annotated data set to (iii) generate and/or train one or more medical models. As used herein, a medical model may refer to a model that is configured to receive one or more inputs related to a medical patient or a medical operation and to generate one or more outputs based on an analysis or an evaluation of the one or more inputs. The one or more outputs generated by the medical model may comprise one or more surgical applications as described below. In some cases, the medical model may be configured to analyze, evaluate, and/or process the inputs by comparing the inputs to other data sets accessible by the medical model. The one or more medical models may be generated using at least the plurality of data inputs, the one or more annotations, and/or the annotated data set. The one or more medical models may be configured to assist a medical operator with performing a surgical procedure. In some cases, aiding the one or more live surgical procedures may comprise providing guidance to a surgeon while the surgeon is performing one or more steps of the one or more live surgical procedures. In some cases, aiding the one or more live surgical procedures may comprise improving a control or a motion of one or more robotic devices that are configured to perform autonomous or semi-autonomous surgery. In some cases, aiding the one or more live surgical procedures may comprise automating one or more steps of a surgical procedure.

The one or more medical models may be trained using the plurality of data inputs, the one or more annotations, the annotated data set, and one or more model training methods. In some cases, the one or more medical models may be trained using neural networks or convolutional neural networks. In some cases, the one or more medical models may be trained using deep learning. In some cases, the deep learning may be supervised, unsupervised, and/or semi-supervised. In some cases, the one or more medical models may be trained using reinforcement learning and/or transfer learning. In some cases, the one or more medical models may be trained using image thresholding and/or color-based image segmentation. In some cases, the one or more medical models may be trained using clustering. In some cases, the one or more medical models may be trained using regression analysis. In some cases, the one or more medical models may be trained using support vector machines. In some cases, the one or more medical models may be trained using one or more decision trees or random forests associated with the one or more decision trees. In some cases, the one or more medical models may be trained using dimensionality reduction. In some cases, the one or more medical models may be trained using one or more recurrent neural networks. In some cases, the one or more recurrent neural networks may comprise a long short-term memory neural network. In some cases, the one or more medical models may be trained using one or more temporal convolutional networks. In some cases, the one or more temporal convolutional networks may have a single or multiple stages. In some cases, the one or more medical models may be trained using data augmentation or generative adversarial networks. In some cases, the one or more medical models may be trained using one or more classical algorithms. The one or more classical algorithms may be configured to implement exponential smoothing, single exponential smoothing, double exponential smoothing, triple exponential smoothing, Holt-Winters exponential smoothing, autoregressions, moving averages, autoregressive moving averages, autoregressive integrated moving averages, seasonal autoregressive integrated moving averages, vector autoregressions, or vector autoregression moving averages.

Using Trained Models to Aid Surgical Procedures

The method may comprise (e) providing the one or more trained medical models to a controller that is in communication with one or more medical devices. In some cases, the one or more medical devices may be configured for autonomous or semi-autonomous surgery. In some cases, the controller may be configured to implement the one or more trained medical models to aid one or more live surgical procedures.

Inputs to Trained Medical Models

The one or more trained medical models may be configured to (i) receive a set of inputs corresponding to the one or more live surgical procedures or one or more surgical subjects of the one or more live surgical procedures and (ii) implement or perform one or more surgical applications, based at least in part on the set of inputs, to enhance a medical operator's ability to perform the one or more live surgical procedures.

In some cases, the set of inputs may comprise medical data associated with the one or more surgical subjects. The one or more surgical subjects may be undergoing the one or more live surgical procedures. The one or more live surgical procedures may be of a same or similar type of surgical procedure as the at least one surgical procedure associated with the plurality of data inputs used to generate and/or train the medical models.

In some cases, the medical data may comprise physiological data of the one or more surgical subjects. The physiological data may comprise an electrocardiogram (ECG or EKG), an electroencephalogram (EEG), an electromyogram (EMG), a blood pressure, a heart rate, a respiratory rate, or a body temperature of the one or more surgical subjects.

In some cases, the medical data may comprise medical imagery. The medical imagery may comprise a pre-operative image selected from the group consisting of an ultrasound image, an X-ray image, an optical coherence tomography (OCT) scan, a computed tomography (CT) scan, a magnetic resonance imaging (MRI) scan, and a positron emission tomography (PET) scan. In some cases, the medical imagery may comprise an intraoperative image of a surgical scene. The intraoperative image may comprise an RGB image, a depth map, a fluoroscopic image, a laser speckle contrast image, a hyperspectral image, a multispectral image, an ultrasound image, and/or a laser doppler image. In some cases, the medical imagery may comprise one or more streams of intraoperative data comprising the intraoperative image. The one or more streams of intraoperative data may comprise a series of intraoperative images obtained successively or sequentially over a time period.

In some cases, the set of inputs may comprise an image or a video of the one or more live surgical procedures. In some cases, the set of inputs may comprise an image or a video of one or more medical instruments used to perform the one or more live surgical procedures.

In some cases, the set of inputs may comprise kinematic data associated with a movement of a robotic device or a medical instrument that is usable to perform one or more steps of the one or more live surgical procedures. The kinematic data may be obtained using an accelerometer or an inertial measurement unit.

In some cases, the set of inputs may comprise user control data corresponding to one or more inputs or motions by the medical operator to control a medical instrument to perform the one or more live surgical procedures.

In some cases, the set of inputs may comprise robotic data associated with a movement or a control of a robotic device to perform one or more steps of the one or more live surgical procedures. The robotic device may comprise a robotic arm that is configured to move or control one or more medical instruments.

In some cases, the set of inputs may comprise kinetic data associated with a force, a stress, or a strain that is exerted on a tissue region of the one or more surgical subjects during the one or more live surgical procedures.

In some cases, the set of inputs may comprise instrument-specific data associated with (i) a physical characteristic of one or more medical instruments used to perform the one or more live surgical procedures or (ii) a functional characteristic associated with an operation or a use of the one or more medical instruments during the one or more live surgical procedures. The physical characteristic may comprise a geometry of the one or more medical instruments.

In some cases, the set of inputs may comprise surgery-specific data associated with the one or more live surgical procedures. The surgery-specific data may comprise information on a type of surgery associated with the one or more live surgical procedures, a plurality of steps associated with the one or more live surgical procedures, one or more timing parameters associated with the plurality of steps, or one or more medical instruments usable to perform the plurality of steps. In some cases, the surgery-specific data may comprise information on at least one of a relative position or a relative orientation of one or more ports through which a medical instrument or an imaging device may be inserted. The one or more ports may correspond to a trocar or an incision on a portion of a subject's body.

In some cases, the set of inputs may comprise subject-specific data associated with the one or more surgical subjects. The subject-specific data may comprise one or more biological parameters of the one or more surgical subjects. In some cases, the one or more biological parameters may correspond to a physical characteristic, a medical condition, or a pathological condition of the one or more surgical subjects. In some cases, the subject-specific data may comprise anonymized or de-identified subject data.

Outputs of the Trained Medical Models

The one or more trained medical models may be configured to (i) receive a set of inputs corresponding to the one or more live surgical procedures or one or more surgical subjects of the one or more live surgical procedures and (ii) implement or perform one or more surgical applications, based at least in part on the set of inputs, to enhance a medical operator's ability to perform the one or more live surgical procedures.

In some cases, the one or more surgical applications comprise image segmentation on one or more images or videos of the one or more live surgical procedures. The image segmentation may be used to identify one or more medical instruments used to perform the one or more live surgical procedures. The image segmentation may be used to identify one or more tissue regions of the one or more surgical subjects undergoing the one or more live surgical procedures. In some cases, the image segmentation may be used to (i) distinguish between healthy and unhealthy tissue regions, or (ii) distinguish between arteries and veins.

In some cases, the one or more surgical applications may comprise object detection for one or more objects or features in one or more images or videos of the one or more live surgical procedures. In some cases, object detection may comprise detecting one or more deformable tissue regions or one or more rigid objects in a surgical scene.

In some cases, the one or more surgical applications may comprise scene stitching to stitch together two or more images of a surgical scene. In some cases, scene stitching may comprise generating a mini map corresponding to the surgical scene. In some cases, scene stitching may be implemented using an optical paintbrush.

In some cases, the one or more surgical applications may comprise sensor enhancement to augment one or more images and/or measurements obtained using one or more sensors with additional information associated with at least a subset of the set of inputs provided to the trained medical models.

In some cases, sensor enhancement may comprise image enhancement. Image enhancement may comprise auto zooming into one or more portions of a surgical scene, auto focus on one or more portions of a surgical scene, lens smudge removal, or an image correction.

In some cases, the one or more surgical applications may comprise generating one or more procedural inferences associated with the one or more live surgical procedures. The one or more procedural inferences may comprise an identification of one or more steps in a surgical procedure or a determination of one or more possible surgical outcomes associated with the performance of one or more steps of a surgical procedure.

In some cases, the one or more surgical applications may comprise registering a pre-operative image of a tissue region of the one or more surgical subjects to one or more live images of the tissue region of the one or more surgical subjects obtained during the one or more live surgical procedures. In some cases, the one or more surgical applications may comprise registering and overlaying two or more medical images. In some cases, the two or more medical images may be obtained or generated using different imaging modalities.

In some cases, the one or more surgical applications may comprise providing an augmented reality or virtual reality representation of a surgical scene. In some cases, the augmented reality or virtual reality representation of the surgical scene may be configured to provide smart guidance for one or more camera operators to move one or more cameras relative to the surgical scene. In other cases, the augmented reality or virtual reality representation of the surgical scene may be configured to provide one or more alternative camera views or display views to a medical operator during the one or more live surgical procedures.

In some cases, the one or more surgical applications may comprise adjusting a position, an orientation, or a movement of one or more robotic devices or medical instruments during the one or more live surgical procedures.

In some cases, the one or more surgical applications may comprise coordinating a movement of two or more robotic devices or medical instruments during the one or more live surgical procedures. The two or more robotic devices may have two or more independently controllable arms. In some cases, the one or more surgical applications may comprise coordinating a movement of a robotic camera and a robotically controlled medical instrument. In some cases, the one or more surgical applications may comprise coordinating a movement of a robotic camera and a medical instrument that is manually controlled by the medical operator.

In some cases, the one or more surgical applications may comprise locating one or more landmarks in a surgical scene. The one or more landmarks may correspond to one or more locations or regions of interest in the surgical scene. In some cases, the one or more landmarks may correspond to one or more critical structures in the surgical scene.

In some cases, the one or more surgical applications may comprise displaying physiological information associated with the one or more surgical subjects on one or more images of a surgical scene obtained during the one or more live surgical procedures.

In some cases, the one or more surgical applications may comprise safety monitoring. In some cases, safety monitoring may comprise geofencing one or more regions in a surgical scene or highlighting one or more regions in the surgical scene for the medical operator to target or avoid.

In some cases, the one or more surgical applications may comprise providing the medical operator with information on an optimal position, orientation, or movement of a medical instrument to perform one or more steps of the one or more live surgical procedures.

In some cases, the one or more surgical applications may comprise informing the medical operator of one or more surgical instruments or surgical methods for performing one or more steps of the one or more live surgical procedures.

In some cases, the one or more surgical applications may comprise informing the medical operator of an optimal stitch pattern.

In some cases, the one or more surgical applications may comprise measuring perfusion, stitch tension, tissue elasticity, or excision margins.

In some cases, the one or more surgical applications may comprise measuring a distance between a first tool and a second tool in real time. In some cases, the distance between the first tool and the second tool may be measured based at least in part on a geometry (e.g., a size and/or a shape) of the first tool and the second tool. In some cases, the distance between the first tool and the second tool may be measured based at least in part on a relative position or a relative orientation of a scope that is used to perform the one or more live surgical procedures.

In some cases, the method may further comprise detecting one or more edges of the first tool and/or the second tool to determine a position and/or an orientation of the first tool relative to the second tool. In some cases, the method may further comprise determining a three-dimensional position of a tool tip of the first tool and a three-dimensional position of a tool tip of the second tool. In some cases, the method may further comprise registering a scope port to a pre-operative image to determine a position and an orientation of the first tool, the second tool, and the scope relative to one or more tissue regions within a surgical patient's body.

In some cases, the one or more detected edges of the tool or the scope may be used to improve position feedback of the tool or the scope. Improving position feedback may enhance an accuracy or a precision with which the tool or the scope is moved (e.g., positioned or oriented relative to the surgical scene) during a surgical procedure. In some cases, a global position or a global orientation of the scope relative to the surgical scene may be obtained using an inertial measurement unit. In some cases, the systems and methods of the present disclosure may be used to detect a global position or a global orientation of one or more tools relative to the surgical scene based at least in part on (i) the global position or global orientation of the scope and (ii) the relative position or relative orientation of the one or more tools in relation to the scope. In some cases, the systems and methods of the present disclosure may be used to determine a depth of camera insertion based at least in part on (i) the global position or the global orientation of the scope, (ii) the global position or the global orientation of the one or more tools, or (iii) the relative position or the relative orientation of the one or more tools in relation to the scope. In some cases, the systems and methods of the present disclosure may be used to determine a depth of tool insertion based at least in part on (i) the global position or the global orientation of the scope, (ii) the global position or the global orientation of the one or more tools, or (iii) the relative position or the relative orientation of the one or more tools in relation to the scope. In some cases, the systems and methods of the present disclosure may be used to predict an imaging region of a camera based at least in part on an estimated or a priori knowledge of a position or an orientation of the camera or a scope port through which the camera is inserted.

In some cases, the one or more surgical applications may comprise measuring a distance between a tool and a scope in real time. In some cases, the distance between the tool and the scope may be measured based at least in part on a geometry (e.g., a size and/or a shapes) of the first tool and the scope. In some cases, the distance between the tool and the scope may be measured based at least in part on a relative position or a relative orientation of the scope. In some cases, the method may further comprise detecting one or more edges of the tool and/or the scope to determine a position and an orientation of the tool relative to the scope. In some cases, the method may further comprise determining a three-dimensional position of a tool tip of the tool and a three-dimensional position of a tip of the scope. In some cases, the method may further comprise registering a scope port to a pre-operative image to determine a position and an orientation of the tool and the scope relative to one or more tissue regions within the surgical patient's body.

In some cases, the one or more surgical applications may comprise displaying one or more virtual representations of one or more tools in a pre-operative image of a surgical scene. In some cases, the one or more surgical applications may comprise displaying one or more virtual representations of one or more medical instruments in a live image or video of a surgical scene.

In some cases, the one or more surgical applications may comprise determining one or more dimensions of a medical instrument that is visible in an image or a video of a surgical scene. In other cases, the one or more surgical applications may comprise determining one or more dimensions of a critical structure of a surgical subject that is visible in an image or a video of a surgical scene.

In some cases, the one or more surgical applications may comprise providing an overlay of a perfusion map and a pre-operative image of a surgical scene. In some cases, the one or more surgical applications may comprise providing an overlay of a perfusion map and a live image of a surgical scene. In some cases, the one or more surgical applications may comprise overlaying a pre-operative image of a surgical scene with a live image of the surgical scene, or overlaying the live image of the surgical scene with the pre-operative image of the surgical scene. The overlay may be provided in real time as the live image of the surgical scene is being obtained during a live surgical procedure.

In some cases, the one or more surgical applications may comprise providing a set of virtual markers to guide the medical operator during one or more steps of the one or more live surgical procedures. The set of virtual markers may indicate where to perform a cut, a stitching pattern, where to move a camera that is being used to monitor a surgical procedure, and/or where to position, orient, or move a medical instrument to optimally perform one or more steps of the surgical procedure.

Validation

In some embodiments, the method may further comprise validating the plurality of data inputs prior to receiving the one or more annotations. Validating the plurality of data inputs may comprise scoring the plurality of data inputs, retaining at least a first subset of the plurality of data inputs with a first set of scores that is above a pre-determined threshold, and discarding at least a second subset of the plurality of data inputs with a second set of scores that is below the pre-determined threshold.

In some cases, the method may further comprise validating the one or more annotations prior to training the medical models. Validating the one or more annotations may comprise scoring the one or more annotations, retaining at least a first subset of the one or more annotations with a first set of scores that is above a pre-determined threshold, and discarding at least a second subset of the one or more annotations with a second set of scores that is below the pre-determined threshold.

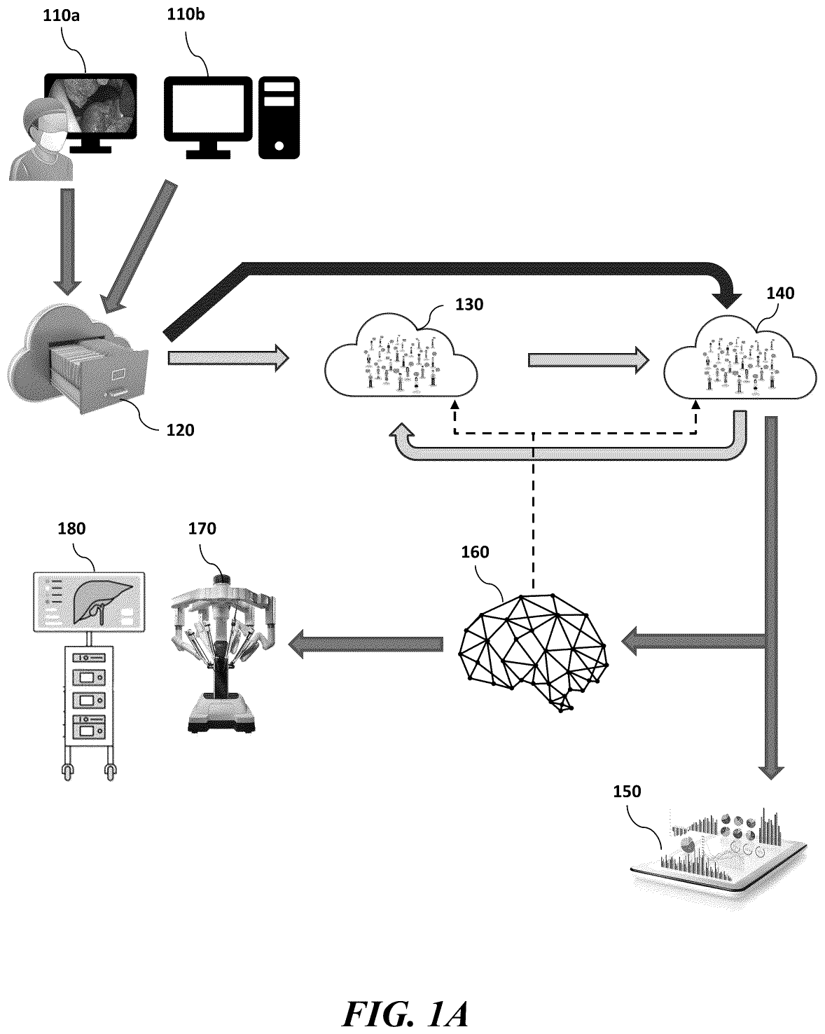

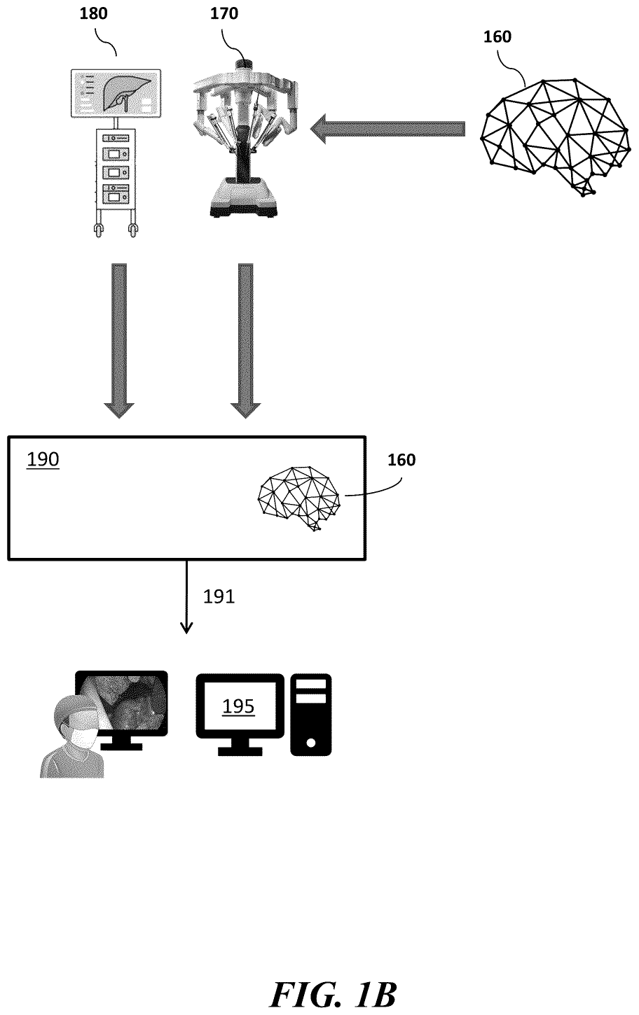

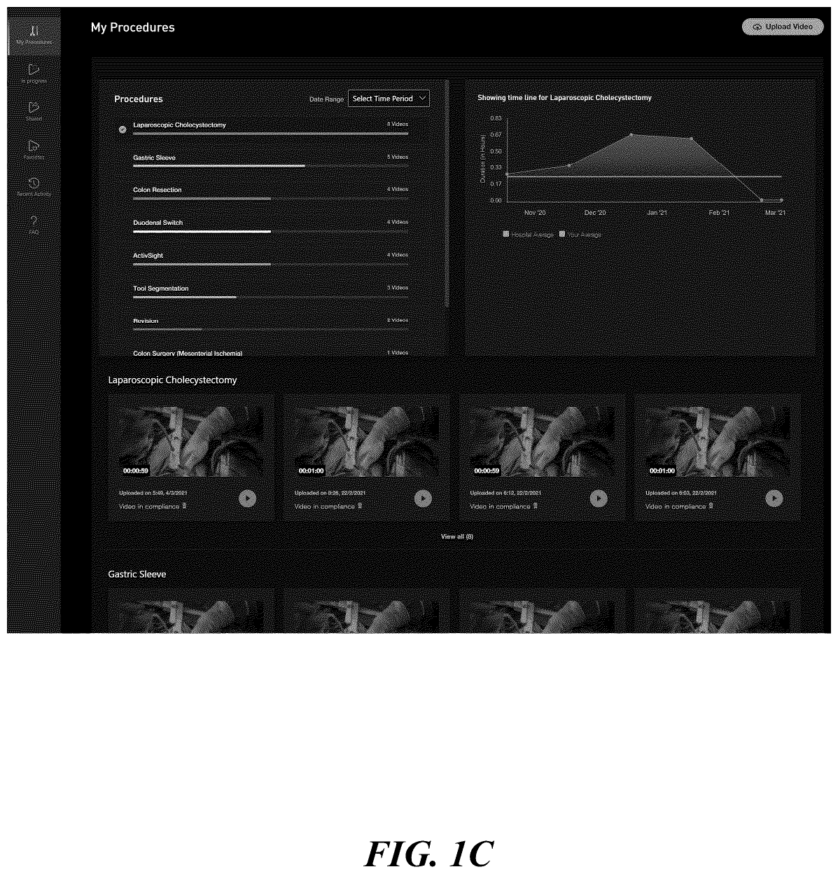

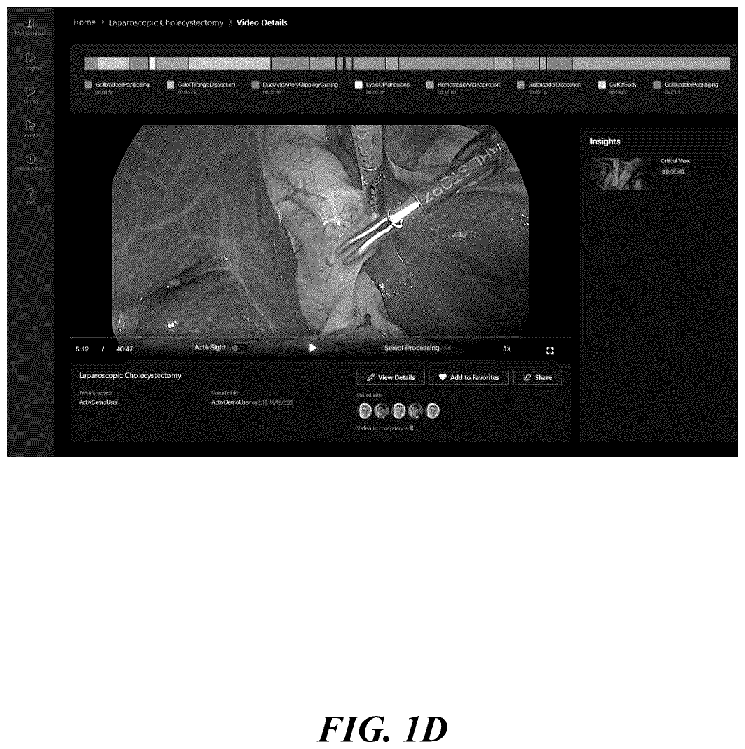

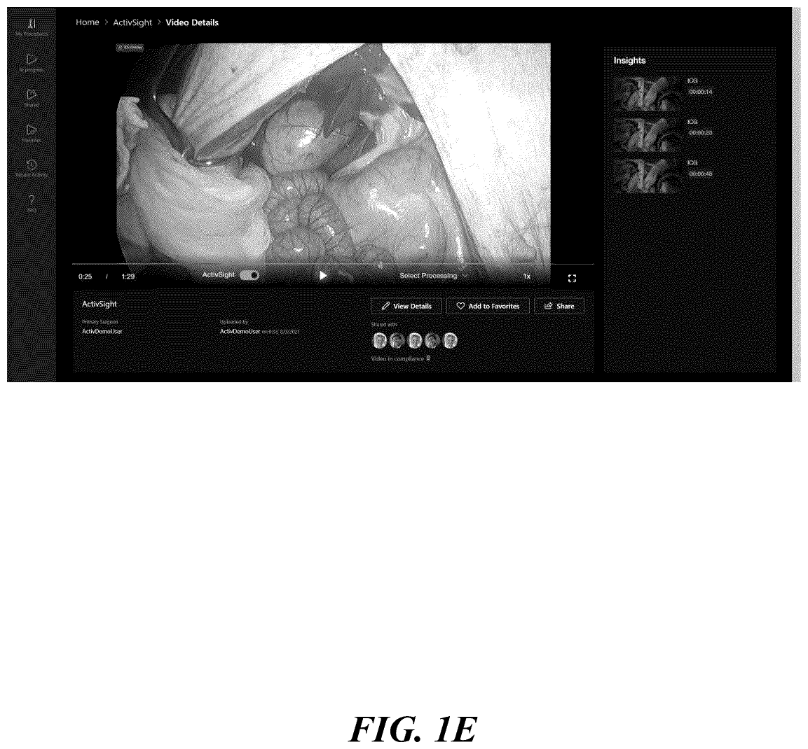

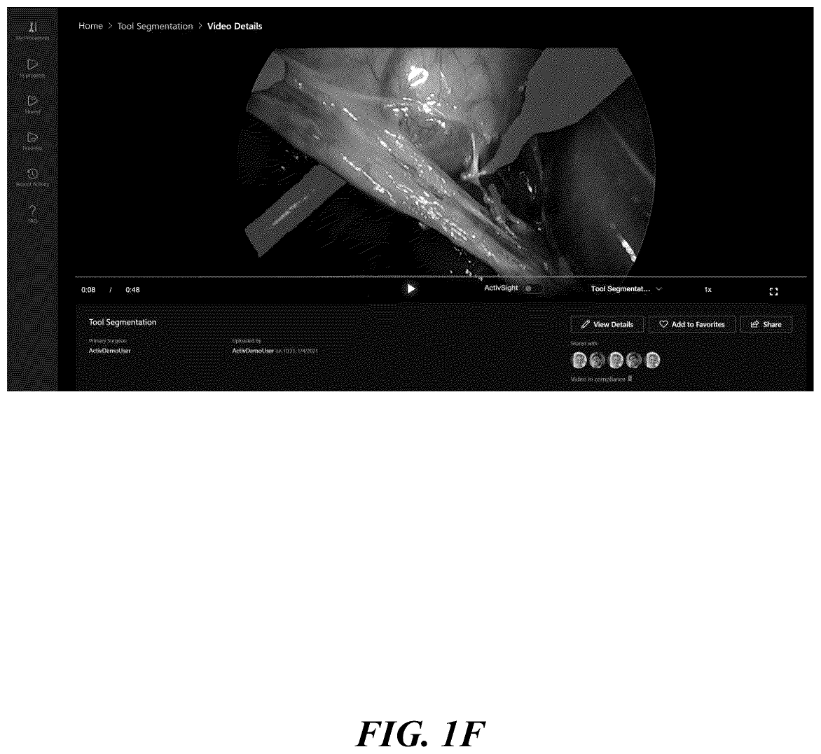

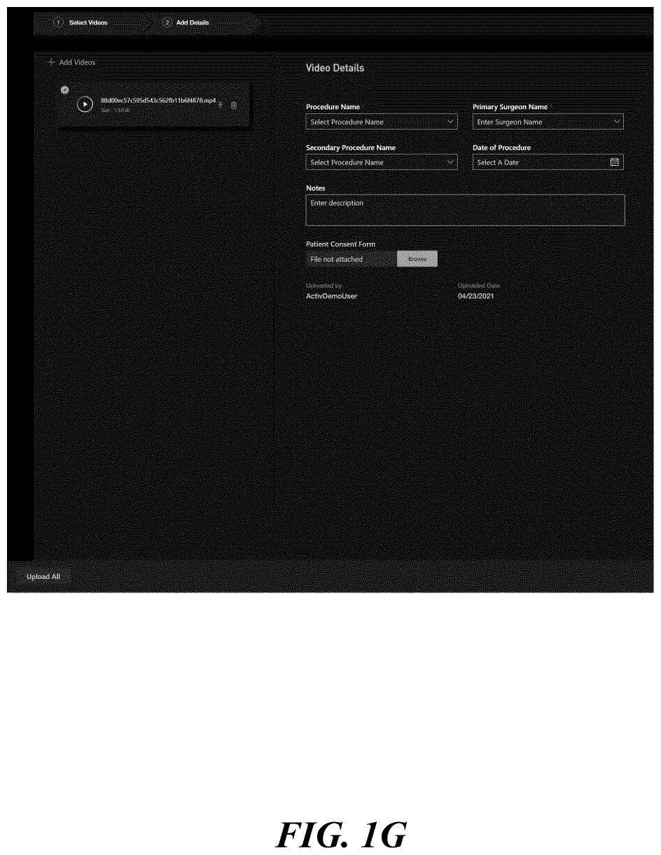

FIG. 1A illustrates a flow diagram for processing medical data. A plurality of data inputs 110a and 110b may be uploaded to a cloud platform 120. In some cases, the plurality of data inputs 110a and 110b may comprise surgical videos of a surgical procedure. The plurality of data inputs 110a and 110b may be uploaded to the cloud platform 120 by a medical device, a health system, a health care facility, a doctor, a surgeon, a healthcare worker, a medical assistant, a scientist, an engineer, a medical device specialist, or a medical device company. The cloud platform 120 may be accessed by one or more data annotators. The data inputs uploaded to the cloud platform 120 may be provided to the one or more data annotators for annotation. The one or more data annotators may comprise generalist crowd annotators 130 and/or expert crowd annotators 140. The generalist crowd annotators 130 and the expert crowd annotators 140 may receive different subsets of the uploaded data based on a level of expertise. Annotation tasks may be assigned based on the annotators' level of expertise. For example, the generalist crowd annotators 130 may be requested to provide non-domain specific annotations, and the expert crowd annotators may be requested to provide domain specific annotations. Annotations generated by the generalist crowd annotators 130 may be provided to the expert crowd annotators 140 for review and quality control. The expert crowd annotators 140 may review the annotations generated by the generalist crowd annotators 130. In some cases, poor quality annotations or incorrection annotations may be sent back to the generalist crowd annotators 130 for re-annotation.

As described above, in some cases, the generalist crowd data annotators 130 may provide non-domain specific annotations for the plurality of data inputs stored one the cloud platform 120. The expert crowd annotators 140 may verify the data uploaded to the cloud platform 120 and/or the data annotations provided by the one or more data annotators 130. Poor quality data or poor quality data annotations may not pass this stage. Poor quality annotations may be sent back to the one or more generalist crowd data annotators 130 for re-annotation. In some cases, poor quality annotations may be sent back to a different group or subset of annotators among the one or more data generalist crowd annotators 130 for re-annotation. Poor quality data or annotations may be filtered out through such a process. In some cases, there may be several levels of data and/or annotation review beyond the review performed by the generalist and expert crowds. For example, there may be three or more levels of data and/or annotation review by three or more distinct groups of annotators. In some cases, the medical data may be annotated by one or more annotators. In some cases, the medical data may be annotated by multiple annotators. Once the data and/or the data annotations have been verified or deemed acceptable for quality assurance purposes, the data and/or the one or more data annotations may be used for data analytics 150. Alternatively, the data and/or the one or more data annotations may be used to generate and/or train one or more medical models 160. The one or more medical models 160 may be deployed through the internet to one or more medical devices 170 or medical systems 180. The one or more medical devices 170 or medical systems 180 may be configured to implement the one or more medical models 160 to provide artificial intelligence (AI) decision support and guidance for medical procedures or analysis of one or more aspects of such medical procedures. In some cases, the one or more medical models 160 may be configured to create annotations for the data uploaded to the cloud platform 120. In some cases, the one or more medical models 160 may be configured to provide one or more annotations as a starting point for the generalist crowd annotators 130 and/or the expert crowd annotators 140. In some cases, the one or more medical models 160 may be configured to verify the one or more annotations provided by the generalist crowd annotators 130 and/or the expert crowd annotators 140.

FIG. 1B illustrates an example of a surgical video processing platform 190 that allows users, medical devices 170, and/or medical systems 180 to upload surgical data to one or more servers (e.g., cloud servers) and to process the surgical data using one or more algorithms or medical models 160 to generate or provide a variety of different insights for the surgical procedure. The one or more algorithms or medical models 160 may be developed and/or trained using annotated data as described elsewhere herein. The annotated data may be generated using any of the data annotation systems and methods described herein. The one or more algorithms or medical models 160 may be used to enhance intra-operative decision making and provide supporting features (e.g., enhanced image processing capabilities or live data analytics) to assist a surgeon during a surgical procedure. In some embodiments, the surgical video processing platform 190 may comprise a cloud based surgical video processing system that can facilitate sourcing of surgical data (e.g., images, videos, and/or audio), process the surgical data, and extract insights from the surgical data.

In some instances, the one or more algorithms or medical models 160 may be implemented live on the medical devices 170 and/or medical systems 180. In such cases, the medical devices 170 and/or medical systems 180 may be configured to process or pre-process medical data (e.g., surgical images or surgical videos) using the one or more algorithms or medical models 160. Such processing or pre-processing may occur in real-time as the medical data is being captured. In other instances, the one or more algorithms or medical models 160 may be used to process the medical data after the medical data is uploaded to the surgical video processing platform 190. In some alternative embodiments, a first set of medical algorithms or models may be implemented on the medical devices 170 and/or medical systems 180, and a second set of medical algorithms or models may be implemented on the back-end of the surgical video processing platform 190 after the medical data is uploaded to the surgical video processing platform 190. The medical data may be processed to generate one or more medical insights 191, which may be provided to one or more users. The one or more users may comprise, for example, a surgeon or a doctor who is performing a surgical procedure or assisting with the surgical procedure.

In some embodiments, the surgical video processing platform 190 may comprise a web portal. The web portal may operate as the platform between the operating room and the one or more medical algorithms or models 160. As described elsewhere herein, the one or more medical algorithms or models 160 may be trained using medical annotation data. Users (e.g., doctors or surgeons who wish to view additional insights 191 relating to a surgical procedure they are currently performing or that they previously performed) may access the web portal view using a computing device 195. The computing device 195 may comprise a computer or a mobile device (e.g., a smartphone or a tablet). The computer device 195 may comprise a display for the user to view one or more surgical videos or one or more insights 191 pertaining to the surgical videos.

In some cases, the surgical video processing platform 190 may comprise a user or web interface that displays a plurality of surgical videos that may be processed to generate or derive one or more medical insights. An example of the user or web interface is illustrated in FIG. 1C. The plurality of surgical videos may comprise surgical videos for procedures that have already been completed, or surgical videos for procedures that are currently ongoing. Users may interact with the user or web interface to select various surgical videos of interest. The plurality of surgical videos may be organized by procedure type, devices used, operator, and/or surgical outcome.

Data Upload

The surgical videos may be uploaded to the surgical video processing platform 190. The surgical videos may be uploaded directly from one or more medical devices, instruments, or systems that are being used to perform or assist with a surgical procedure. In some cases, the surgical videos may be captured using the one or more medical devices, instruments, or systems. The surgical videos may be anonymized before or after being uploaded to the surgical video processing platform 190 to protect the privacy of the subject or patient. In some cases, the anonymized and de-identified data may be provided to various annotators for annotations, and/or used to train various medical algorithms or models as described elsewhere herein. In some cases, de-identification may be performed in real time as the medical data is being received, obtained, captured, or processed.

In some cases, the surgical data or surgical videos may be uploaded automatically by the one or more medical devices, instruments, or systems. The one or more medical devices, instruments, or systems may need to be enrolled, validated, provisioned, and/or authorized in order to connect with the surgical video processing platform 190 and to send or receive data from the surgical video processing platform 190.

In some cases, the one or more medical devices, instruments, or systems may be enrolled based on a whitelist that is created or managed by a device manufacturer, a healthcare facility in which a surgical procedure is being performed, a doctor or a surgeon performing the surgical procedure, or any other medical worker of the healthcare facility. The medical devices, instruments, or systems may have an associated identifier that can be used to verify and validate the devices, instruments, or systems to facilitate enrollment with a device provisioning service. In some cases, the devices, instruments, or systems may be configured to perform auto enrollment.

In some cases, the one or more medical devices, instruments, or systems may be provisioned (i.e., registered with the device provisioning service). Further, the one or more medical devices, instruments, or systems may be assigned to a designated hub and/or authorized to communicate with the hub or the surgical video processing platform 190 directly. In some cases, the designated hub may be used to facilitate communications or data transfer between a video processing system of the surgical video processing platform 190 and the one or more medical devices, instruments, or systems. Once registered and authorized, the one or more medical devices, instruments, or systems may be configured to automatically upload medical data and/or surgical videos to the video processing system via the hub.

Alternatively, the surgical data or surgical videos may be uploaded manually by a user (e.g., a doctor or a surgeon). FIG. 1G shows an example of a user interface for manually uploading surgical data. The user interface may permit an uploader to provide additional contextual data corresponding to the surgical data or the surgical procedure captured in the surgical video. The additional contextual data may comprise, for example, procedure name, procedure type, surgeon name, surgeon ID, date of procedure, medical information associated with the patient, or any other information relating to the surgical procedure. The additional contextual data may be provided in the form of one or more user-provided inputs. Alternatively, the additional contextual data may be provided or derived from one or more electronic medical records associated with one or more medical or surgical procedures and/or one or more patients or medical subjects who have undergone a medical or surgical procedure, or will be undergoing a medical or surgical procedure. The surgical video processing platform 190 may be configured to determine which medical algorithms or models to use to process or post-process the surgical data or surgical videos, based on the one or more inputs provided by the uploader.

Medical Insights

The surgical videos may be processed to generate one or more insights. In some cases, the surgical videos may be processed on the medical devices, instruments, or systems before being uploaded to the surgical video processing platform 190. In other cases, the surgical videos may be processed after being uploaded to the surgical video processing platform 190. Processing the surgical videos may comprise applying one or more medical algorithms or models 160 to the surgical videos to determine one or more features, patterns, or attributes of the medical data in the surgical videos. In some cases, the medical data may be classified, segmented, or further analyzed based on the features, patterns, or attributes of the medical data. The medical algorithms or models 160 may be configured to process the surgical videos based on a comparison of the medical data in the surgical videos to medical data associated with other reference surgical videos. The other reference surgical videos may correspond to surgical videos for other similar procedures. In some cases, the reference surgical videos may comprise one or more annotations provided by various medical experts and/or specialists.

In some cases, the medical algorithms or models may be implemented in real-time as the medical data or the surgical video is being captured. In some cases, the medical algorithms or models may be implemented live on the tool, device, or system that is capturing the medical data or the surgical video. In other cases, the medical algorithms or models may be implemented on the back-end of the surgical video processing platform 190 after the medical data or the surgical video is uploaded to the web platform. In some cases, the medical data or the surgical video may be pre-processed on the tool, device, or system, and post-processed in the back-end after being uploaded. Such post-processing may be performed based on one or more outputs or associated data sets generated during the pre-processing phase.

In some cases, the medical algorithms or models may be trained using annotated data. In other cases, the medical algorithms or models may be trained using unannotated data. In some embodiments, the medical algorithms or models may be trained using a combination of annotated data and unannotated data. In some cases, the medical algorithms or models may be trained using supervised learning and/or unsupervised learning. In other cases, the medical algorithms or models may not or need not be trained. The insights generated for the surgical videos may be generated using medical algorithms or models that have been trained using annotated data. Alternatively, the insights generated for the surgical videos may be generated using medical algorithms or models that have not been trained using annotated data, or that do not require training.

In some cases, the medical algorithms or models may comprise algorithms or models for tissue tracking. Tissue tracking may comprise tracking a movement or a deformation of a tissue in a surgical scene. In some cases, the algorithms or models may be used to provide depth information from stereo images, RGB data, RGB-D image data, or time of flight data. In some cases, the algorithms or models may be implemented to perform deidentification of medical data or patient data. In some cases, the algorithms or models may be used to perform tool segmentation, phase of surgery breakdown, critical view detection, tissue structure segmentation, and/or feature detection. In some cases, the algorithms or models may provide live guidance based on the detection of one or more tools, surgical phases, features (e.g., biological, anatomical, physiological, or morphological features), critical views, or movements of tools or tissues in or near the surgical scene. In some cases, the algorithms or models may identify and/or track the locations of certain structures as the surgeon is performing a surgical task near such structures. In some cases, the algorithms or models may be used to generate synthetic data, for example, synthetic ICG images, for simulation and/or extrapolation. In some cases, the algorithms or models may be used for image quality assessment (e.g., is an image blurry due to motion or imaging parameters). In some cases, the algorithms or models may be used to provide one or more surgical inferences (e.g., is a tissue alive or not alive, where to cut, etc.).

In some cases, the insights may comprise a timeline of a surgical procedure. The timeline may comprise a temporal breakdown of the surgical procedure by surgical step or surgical phase, as shown in FIG. 1D. The temporal breakdown may comprise a color coding for the different surgical steps or phases. A user may interact with the timeline to view or skip to one or more surgical phases of interest. In some cases, the timeline may comprise one or more timestamps corresponding to when certain imaging modalities were turned on or off. The timestamps may be provided by the device capturing the surgical video or may be generated using one or more post-processing methods (e.g., by processing the medical data or surgical video using the one or more medical algorithms or models). In some cases, the timestamps may be manually marked by a user. For example, the user may use an input device (e.g., a mouse, a touchpad, a stylus, or a touchscreen) to mark the one or more timestamps. In some cases, the user may provide an input (e.g., a touch, a click, a tap, etc.) to designate one or more time points of interest while observing the surgical video data. In some cases, one or more algorithms may be used to recognize the inputs and translate them into one or more timestamps.

In some cases, the insights may comprise an insights bar. The insights bar may comprise a link, a timestamp, or a labeled window or timepoint that indicates when a critical view of safety is achieved. A user may interact with the various links, timestamps, and/or labeled windows or timepoints to view one or more portions of a surgical video corresponding to the critical view.

In some cases, the insights may comprise augmented visualization by way of image or video overlays, or additional video data corresponding to different imaging modalities. As shown in FIG. 1E, the platform may provide the user with the option to select various types of image processing, and to select various types of imaging modalities or video overlays for viewing. In some examples, the imaging modalities may comprise, for example, RGB imaging, laser speckle imaging, time of flight depth imaging, ICG fluorescence imaging, tissue autofluorescence imaging, or any other type of imaging using a predetermined range of wavelengths. The video overlays may comprise, in some cases, perfusion views and/or ICG fluorescence views. Such video overlays may be performed in real-time, or may be implemented after the surgical videos are pre-processed using the one or more medical algorithms or models described elsewhere herein. In some cases, the algorithms or models may be run on the video and the processed video data may be saved, and the overlay corresponding to the processed video data may then be performed live when a user toggles the overlay using one or more interactive user interface elements (e.g., buttons or toggles) provided by the surgical video processing platform 190. The various types of imaging modalities and the corresponding visual overlays may be toggled on and off by the user as desired (e.g., by clicking a button or a toggle). In some cases, one or more processed videos may be saved (e.g., to local storage or cloud storage), and a user may toggle between the one or more processed videos. For example, the surgical video may be processed to generate a first processed video corresponding to a first imaging modality and a second processed video corresponding to a second imaging modality. The user may view the first processed video for a first portion of the surgical procedure, and switch or toggle to the second processed video for a second portion of the surgical procedure.

In some cases, the insights may comprise tool segmentation as shown in FIG. 1F. Tool segmentation may permit a user to view and track a tool that is being used to perform one or more steps of the surgical procedure. The tracking of the tool may be performed visually and/or computationally (i.e., the coordinates of the tool in three-dimensional space may be tracked, or a position and/or orientation of the tool may be tracked relative to a scope or relative to one or more tissue regions in a surgical scene).

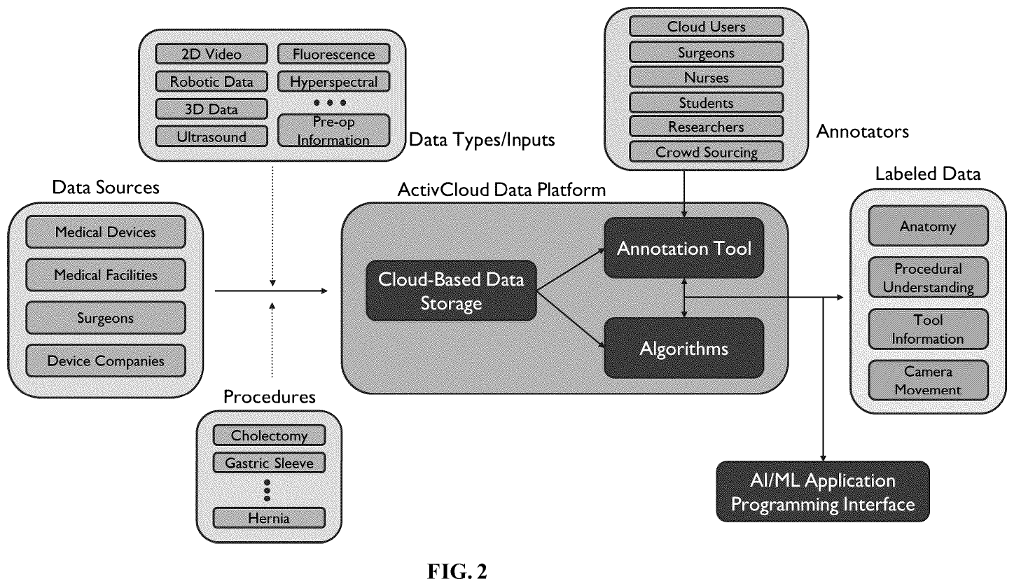

FIG. 2 illustrates a flow diagram for annotating medical data. A plurality of data sources 210 may be leveraged to generate and/or compile a plurality of data inputs 220. The plurality of data sources 210 may comprise medical devices, medical facilities, surgeons, and/or medical device companies. The plurality of data inputs 220 may comprise two-dimensional (2D) video, robotic data, three-dimensional (3D) data such as depth information associated with one more medical images, ultrasound data, fluorescence data, hyperspectral data, and/or pre-operative information associated with one or more medical patients or surgical subjects. The plurality of data inputs 220 may be associated with one or more procedures 230. The one or more procedures 230 may comprise, for example, a colectomy, a gastric sleeve surgery, a surgical procedure to treat or repair a hernia, or any other type of surgical procedure as described elsewhere herein. The plurality of data inputs may be provided to a cloud data platform 240. The cloud data platform 240 may comprise cloud-based data storage for storing the plurality of data inputs 220. The cloud data platform 240 may be configured to provide one or more data annotators 250 with access to an annotation tool. The one or more data annotators 250 may comprise surgeons, nurses, students, medical researchers, and/or any end users with access to the cloud server or platform for annotation based on crowd sourcing. The annotation tool may be used to annotate and/or label the plurality of data inputs 220 to generate labeled or annotated data 260. The annotation tool may be used to annotate and/or label the plurality of data inputs 220 with aid of one or more data annotation algorithms in order to generate the annotated data 260. The annotated data 260 may comprise labeled data associated with an anatomy of a medical patient or surgical subject, a procedural understanding, tool information, and/or camera movement. The annotated data 260 may be provided to an artificial intelligence (AI) or machine learning (ML) application program interface 270 to generate one or more medical models as described elsewhere herein.

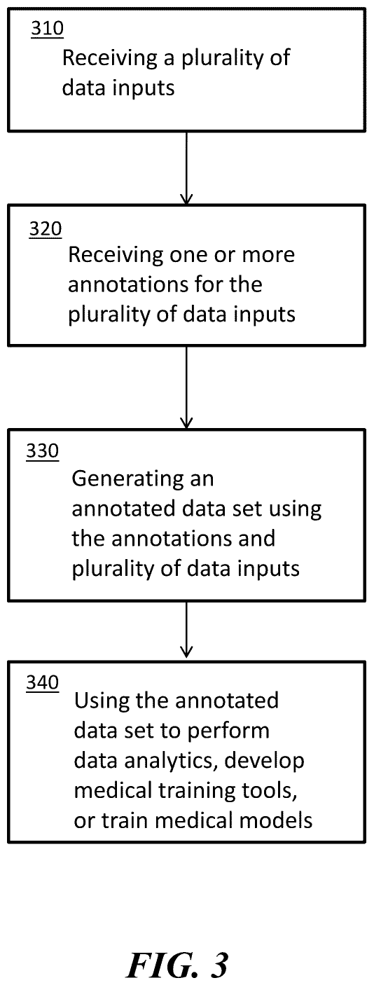

FIG. 3 illustrates an exemplary method for processing medical data. The method may comprise a step 310 comprising (a) receiving a plurality of data inputs associated with (i) at least one medical patient or (ii) at least one surgical procedure. The method may comprise another step 320 comprising (b) receiving one or more annotations for at least a subset of the plurality of data inputs. The method may comprise another step 330 comprising (c) generating an annotated data set using (i) the one or more annotations and (ii) one or more data inputs of the plurality of data inputs. The method may comprise another step 340 comprising (d) using the annotated data set to (i) perform data analytics for the plurality of data inputs, (ii) develop one or more medical training tools, or (iii) train one or more medical models.

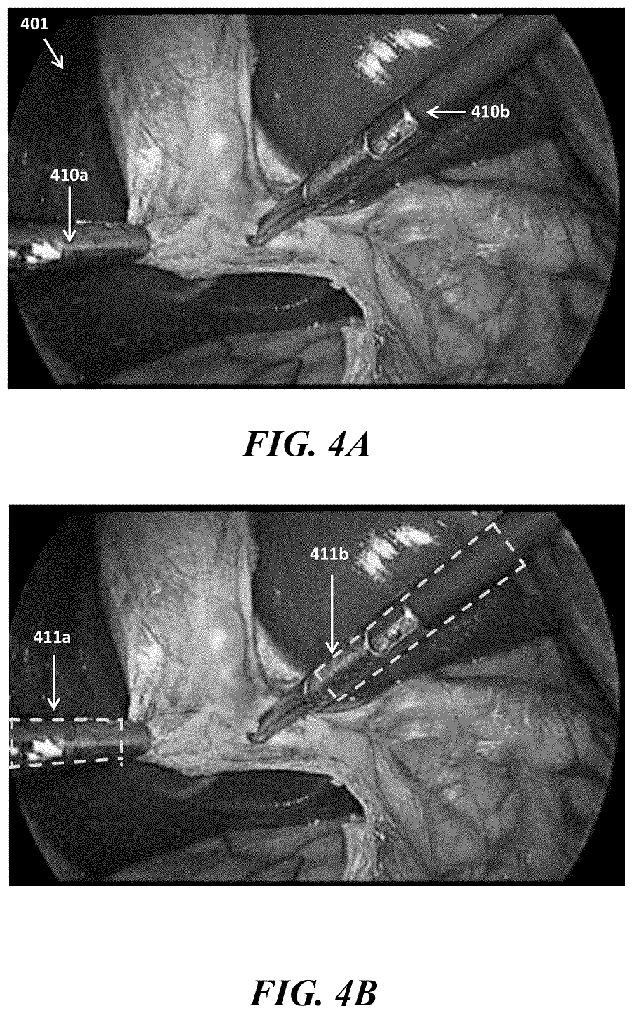

FIG. 4A illustrates a surgical video that may be captured of a surgical scene 401 during a surgical procedure. The surgical video may comprise a visualization of a plurality of surgical tools 410a and 410b. As shown in FIG. 4B, the one or more medical models described elsewhere herein may be used to detect one or more tool edges 411a and 411b of the one or more medical tools 410a and 410b.

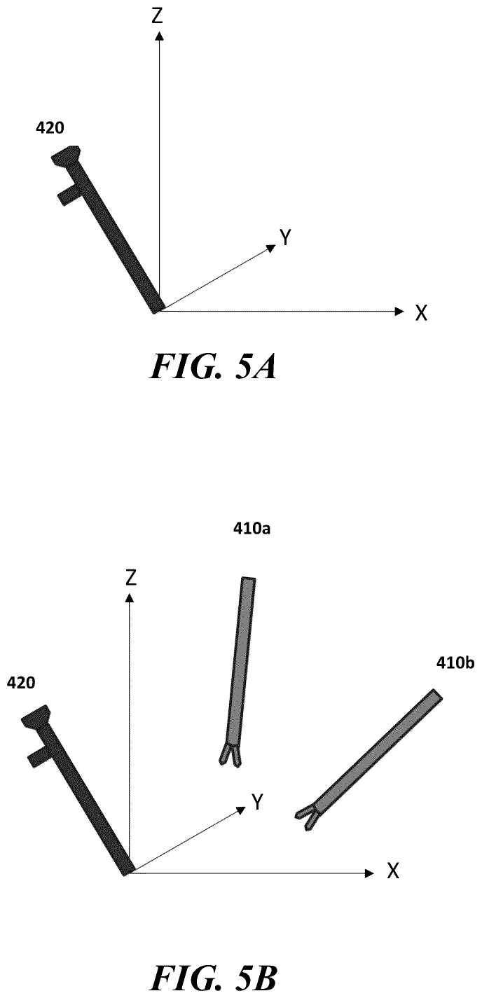

FIG. 5A illustrates a position and an orientation of a scope 420 relative to the surgical scene. The position and orientation of the scope 420 relative to the surgical scene may be derived from the surgical video illustrated in FIG. 4A and FIG. 4B. The position and orientation of the scope 420 relative to the surgical scene may be derived using an inertial measurement unit. As shown in FIG. 5B, the position and the orientation of the surgical tools 410a and 410b relative to the scope 420 may also be derived in part based on the detected tool edges 411a and 411b illustrated in FIG. 4B.

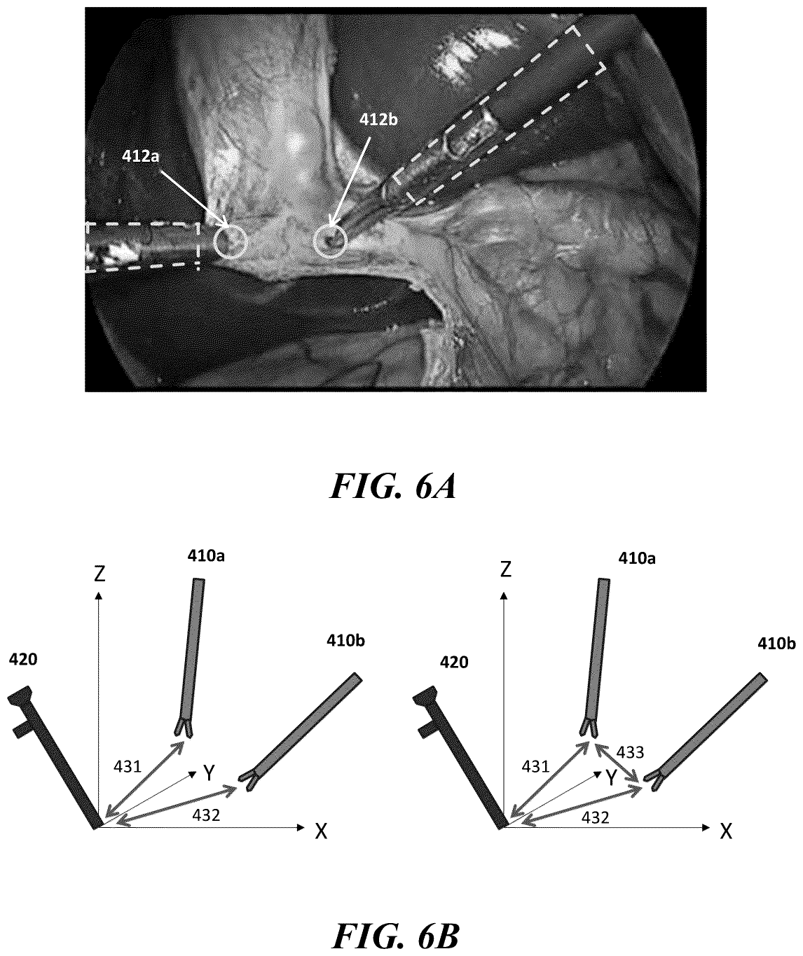

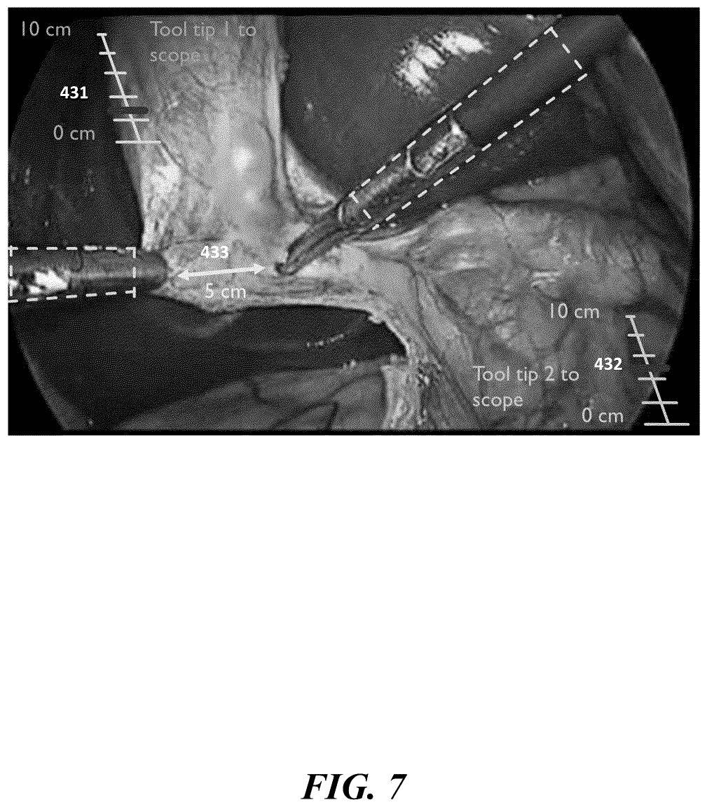

FIG. 6A illustrates a plurality of tool tips 412a and 412b detected within a surgical video of the surgical scene. The plurality of tool tips 412a and 412b may be associated with the plurality of medical tools illustrated in FIG. 4A and FIG. 4B. As shown in FIG. 6B, the position of the tool tips 412a and 412b may be used in combination with the detected tool edges and a known diameter of the plurality of surgical tools to estimate a three-dimensional (3D) position of the tool tips 412a and 412b relative to the scope 420. The position of the tool tips 412a and 412b may be used in combination with the detected tool edges and a known diameter of the plurality of surgical tools to estimate a distance 431 and 432 between the scope 420 and the one or more medical tools 410a and 410b. In some cases, the position of the tool tips 412a and 412b may be used in combination with the detected tool edges and a known diameter of the plurality of surgical tools to estimate a distance 433 between the tool tips 412a and 412b of the one or more medical tools 410a and 410b. FIG. 7 illustrates an augmented reality view of the surgical scene showing a tip-to-tip distance 433 between the one or more medical tools and tip-to-scope distances 431 and 432 between the scope and the one or more medical tools. The tip-to-tip distance 433 between the one or more medical tools and the tip-to-scope distances 431 and 432 between the scope and the one or more medical tools may be computed and/or updated in real-time as the surgical video of the surgical scene is being captured or obtained.

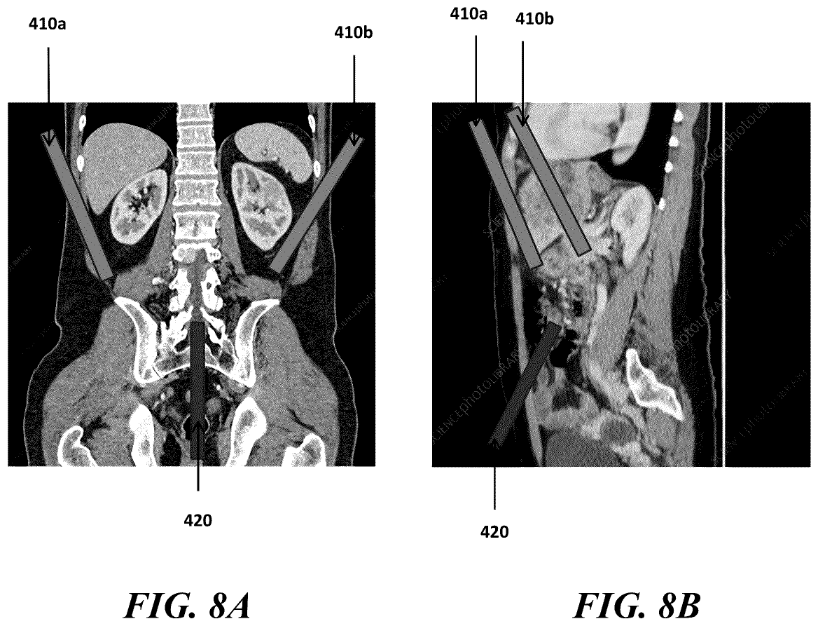

As shown in FIG. 8A and FIG. 8B, in some cases a scope port associated with the scope 420 may be registered to a CT image of the patient to provide one or more virtual views of the one or more medical tools 410a and 410b inside the patient. The one or more virtual views of the one or more medical tools 410a and 410b inside the patient may be computed and/or updated in real-time as the surgical video of the surgical scene is being captured or obtained.

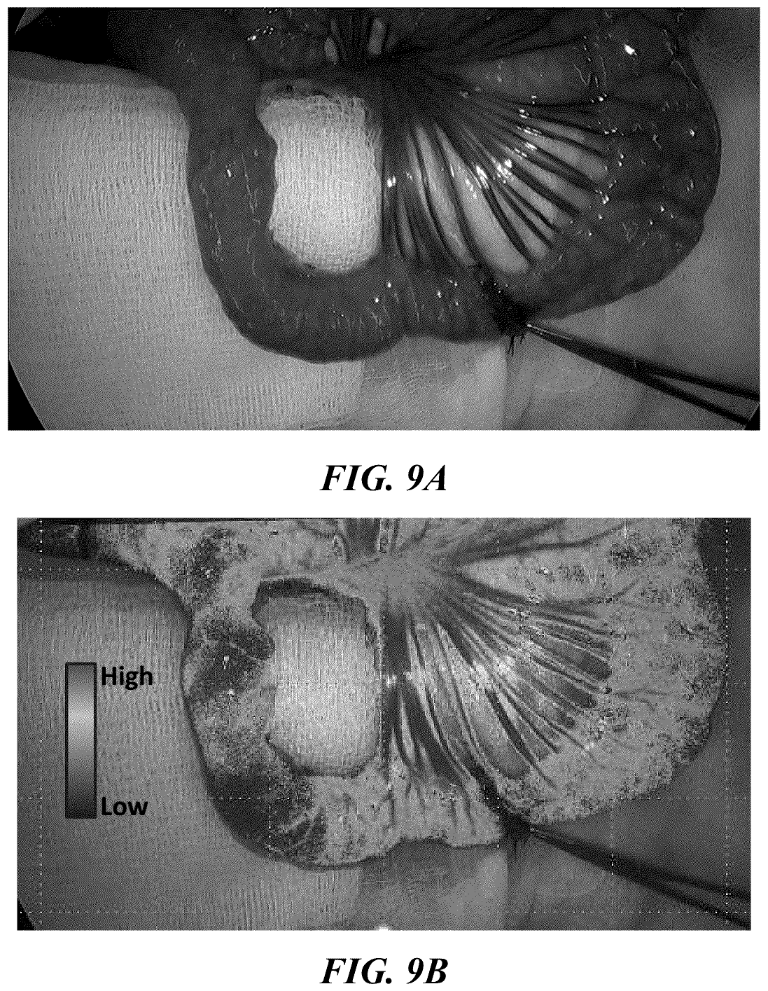

FIG. 9A illustrates a surgical video of a tissue region of a patient. As shown in FIG. 9B, the one or more medical models described herein may be implemented on a medical imaging system to provide RGB and perfusion data associated with the tissue region of the patient. The one or more medical models implemented on the medical imaging system may provide a visualization of high flow areas within the tissue region, and may indicate tissue viability in real-time as the surgical video of the tissue region is being captured or obtained.

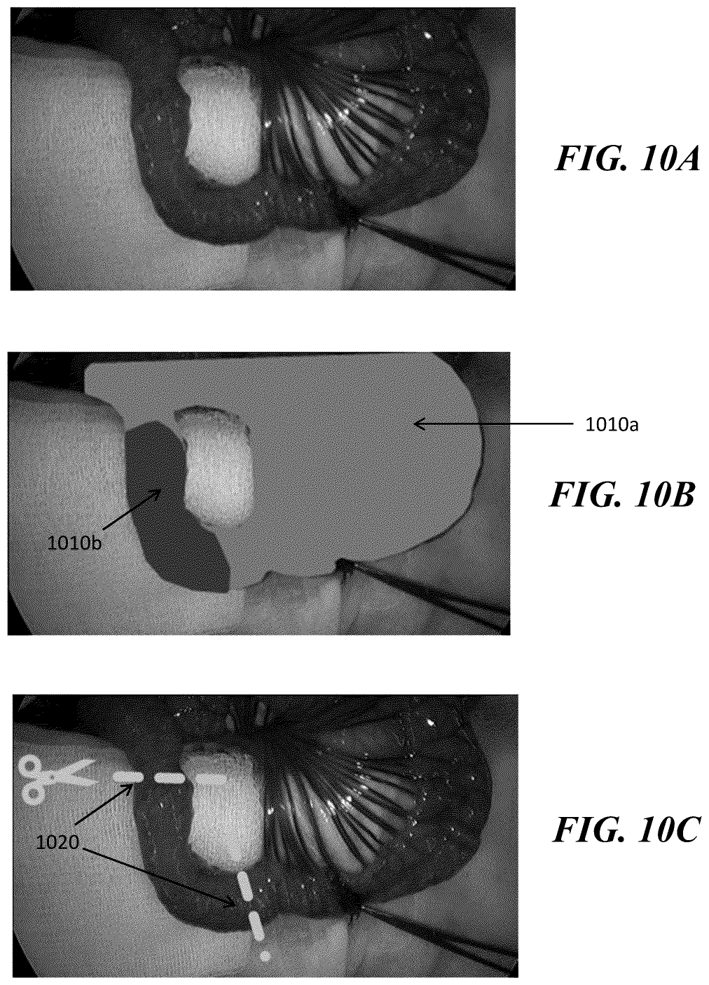

FIG. 10A illustrates a surgical video of a tissue region of a medical patient or surgical subject. FIG. 10B illustrates annotated data that may be generated based on one or more annotations 1010a and 1010b provided by one or more annotators for a surgical video of a tissue region of a medical patient or surgical subject. The one or more annotations 1010a and 1010b may be overlaid on the surgical video of the tissue region of the subject. The one or more medical models described herein may be implemented to provide a real-time display of augmented visuals and surgical guidance, such as virtual markings 1020 indicating to a surgical operator where to make a cut, as shown in FIG. 10C.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

### Computer Systems

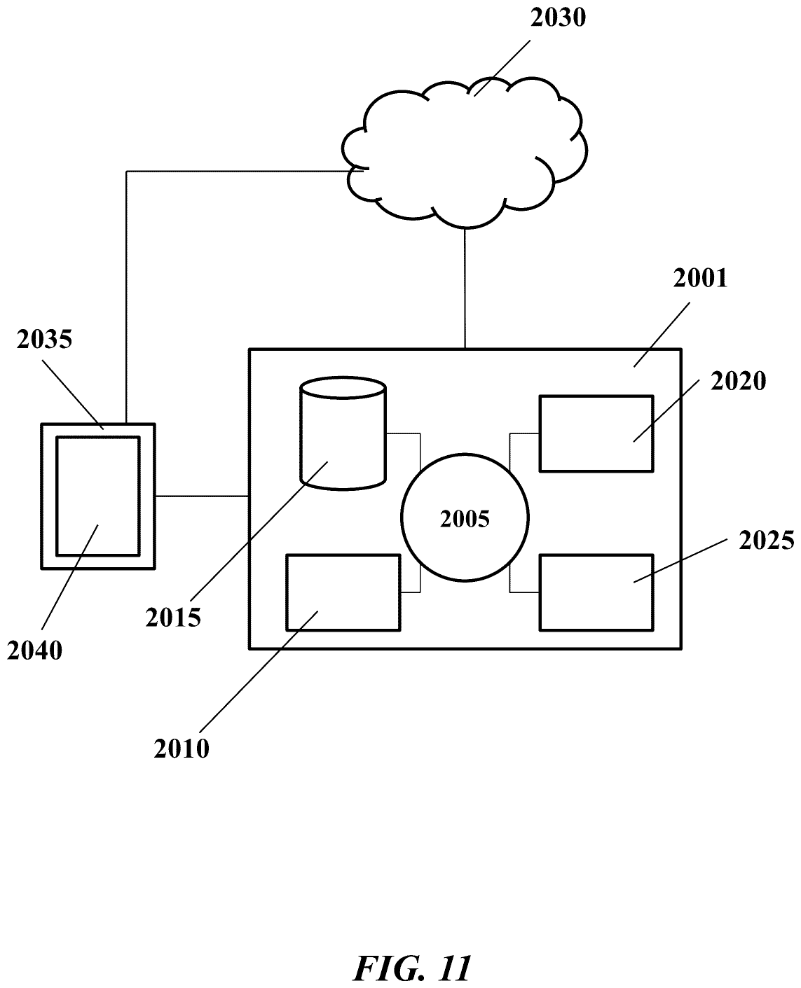

In another aspect, the present disclosure provides computer systems that are programmed or otherwise configured to implement methods of the disclosure, e.g., any of the subject methods for processing medical data. FIG. 11 shows a computer system 2001 that is programmed or otherwise configured to implement a method for processing medical data. The computer system 2001 may be configured to, for example, (a) receive a plurality of data inputs associated with (i) at least one medical patient or (ii) at least one surgical procedure; (b) receive one or more annotations for at least a subset of the plurality of data inputs; (c) generate an annotated data set using (i) the one or more annotations and (ii) one or more data inputs of the plurality of data inputs; and (d) use the annotated data set to (i) perform data analytics for the plurality of data inputs, (ii) develop one or more medical training tools, or (iii) train one or more medical models. The computer system 2001 can be an electronic device of a user or a computer system that is remotely located with respect to the electronic device. The electronic device can be a mobile electronic device.

The computer system 2001 may include a central processing unit (CPU, also “processor” and “computer processor” herein) 2005, which can be a single core or multi core processor, or a plurality of processors for parallel processing. The computer system 2001 also includes memory or memory location 2010 (e.g., random-access memory, read-only memory, flash memory), electronic storage unit 2015 (e.g., hard disk), communication interface 2020 (e.g., network adapter) for communicating with one or more other systems, and peripheral devices 2025, such as cache, other memory, data storage and/or electronic display adapters. The memory 2010, storage unit 2015, interface 2020 and peripheral devices 2025 are in communication with the CPU 2005 through a communication bus (solid lines), such as a motherboard. The storage unit 2015 can be a data storage unit (or data repository) for storing data. The computer system 2001 can be operatively coupled to a computer network (“network”) 2030 with the aid of the communication interface 2020. The network 2030 can be the Internet, an internet and/or extranet, or an intranet and/or extranet that is in communication with the Internet. The network 2030 in some cases is a telecommunication and/or data network. The network 2030 can include one or more computer servers, which can enable distributed computing, such as cloud computing. The network 2030, in some cases with the aid of the computer system 2001, can implement a peer-to-peer network, which may enable devices coupled to the computer system 2001 to behave as a client or a server.

The CPU 2005 can execute a sequence of machine-readable instructions, which can be embodied in a program or software. The instructions may be stored in a memory location, such as the memory 2010. The instructions can be directed to the CPU 2005, which can subsequently program or otherwise configure the CPU 2005 to implement methods of the present disclosure. Examples of operations performed by the CPU 2005 can include fetch, decode, execute, and writeback.

The CPU 2005 can be part of a circuit, such as an integrated circuit. One or more other components of the system 2001 can be included in the circuit. In some cases, the circuit is an application specific integrated circuit (ASIC).

The storage unit 2015 can store files, such as drivers, libraries and saved programs. The storage unit 2015 can store user data, e.g., user preferences and user programs. The computer system 2001 in some cases can include one or more additional data storage units that are located external to the computer system 2001 (e.g., on a remote server that is in communication with the computer system 2001 through an intranet or the Internet).

The computer system 2001 can communicate with one or more remote computer systems through the network 2030. For instance, the computer system 2001 can communicate with a remote computer system of a user (e.g., a healthcare provider, a doctor, a surgeon, a medical assistant, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2001 via the network 2030.

Methods as described herein can be implemented by way of machine (e.g., computer processor) executable code stored on an electronic storage location of the computer system 2001, such as, for example, on the memory 2010 or electronic storage unit 2015. The machine executable or machine readable code can be provided in the form of software. During use, the code can be executed by the processor 2005. In some cases, the code can be retrieved from the storage unit 2015 and stored on the memory 2010 for ready access by the processor 2005. In some situations, the electronic storage unit 2015 can be precluded, and machine-executable instructions are stored on memory 2010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 2001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2001 can include or be in communication with an electronic display 2035 that comprises a user interface (UI) 2040 for providing, for example, a portal for a surgical operator to view one or more portions of a surgical scene using augmented visualizations that are generated using the one or more medical models described herein. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2005. The algorithm may be configured to (a) receive a plurality of data inputs associated with (i) at least one medical patient or (ii) at least one surgical procedure; (b) receive one or more annotations for at least a subset of the plurality of data inputs; (c) generate an annotated data set using (i) the one or more annotations and (ii) one or more data inputs of the plurality of data inputs; and (d) use the annotated data set to (i) perform data analytics for the plurality of data inputs, (ii) develop one or more medical training tools, or (iii) train one or more medical models.

Virtual Surgical Assistant

In another aspect, the present disclosure provides systems and methods for providing virtual surgical assistance. One or more virtual surgical assistants may be used to provide the virtual surgical assistance. The virtual surgical assistant may be an artificial intelligence or machine learning based entity that is configured to aggregate surgical or medical knowledge from world renowned experts and deliver the aggregated surgical or medical knowledge into an operating room. The knowledge can be built on various information sources, such as surgical video data and electronic medical records data, combined with expert annotations. The virtual surgical assistant may be configured to deliver useful insights to surgeons and surgical staff in real time before, during, and/or after medical procedures. Such insights may be delivered in a timely manner with high confidence and accuracy to provide effective clinical support. In some cases, the virtual surgical assistant may be implemented using one or more medical algorithms or medical models as described elsewhere herein.

In some cases, the virtual surgical assistant may provide advanced visualization data for a surgical procedure on a screen or display located in an operating room. The virtual surgical assistant may be used for collaborative robotics or to facilitate collaboration between a human operator and a robotic system (e.g., a robotic system for performing or assisting one or more medical or surgical procedures).

One motivation for developing virtual surgical assistants is that the number of deaths due to avoidable surgical complication is rather high, and about a quarter of the medical errors that occur during a surgical procedure are preventable. Virtual surgical assistants can be used to provide useful and timely information during surgeries to save lives and improve surgical outcomes. Another important motivation is that surgical care access around the globe is heterogenous. Billions of people have limited or minimal access to surgical care, and even when access is available, the lack of medical or surgical expertise, particularly for complicated procedures, can increase the number of preventable surgical errors that occur during a procedure. A virtual surgical assistant that is present in an operating room and/or accessible to medical workers in the operation room can help to provide additional medical or surgical insights which can reduce an occurrence or severity of errors during a procedure.

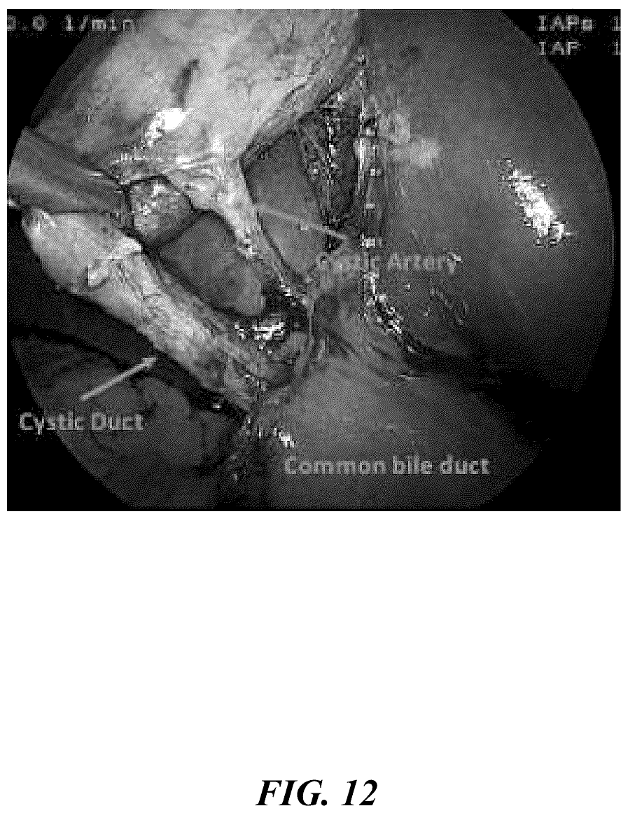

The virtual surgical assistant may be developed or trained based on an identified need. The identified need may correspond to certain procedures where the number of preventable errors and associated human and material costs is rather large, which indicates that there is room for improvement with respect to the performance or execution of such procedures. For example, in the case of laparoscopic cholecystectomies, while the number of bile duct injuries is only about 0.3%, the complications can be life altering. FIG. 12 illustrates the critical view of safety during a laparoscopic cholecystectomy. This view can be used to indicate or verify that no critical structures, such as a common bile duct, are in danger of being damaged. As a surgeon or doctor performs the laparoscopic cholecystectomy, a virtual surgical assistant may be used to identify a presence or an absence of certain critical structures, and to inform the surgeon of any risks of damaging the critical structures as the surgeon operates on or near the critical structures.

After identifying one or more candidate procedures that can benefit from virtual surgical assistance, the best approaches, techniques, and/or methods for performing the respective candidate procedures may be determined. The virtual surgical assistants may be trained to recognize a surgical procedure that is similar to a candidate procedure, and to provide guidance that tracks the best approaches, techniques, and/or methods for performing the respective candidate procedures. In some cases, the virtual surgical assistant can be configured to provide guidance for a variety of surgical tasks. In other cases, the virtual surgical assistant may be a highly specialized entity that can provide guidance specific to a particular step within a procedure. In any case, the virtual surgical assistant may be trained using the collective knowledge and experience of multiple entities and/or institutions with advanced expertise in various surgical procedures (e.g., academic institutions, universities, research centers, medical centers, hospitals, etc.).

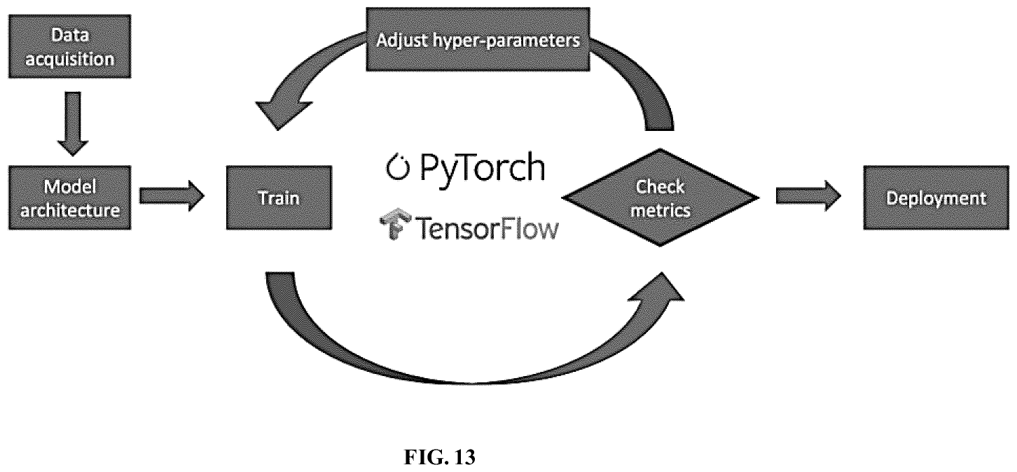

FIG. 13 illustrates an example of a machine learning development pipeline for training and deploying one or more virtual surgical assistants. Training machine learning based solutions may generally involve acquiring medical or surgical data while investigating various model architectures. When a specific architecture is picked and enough data is collected, iterative training may be performed using various strategies and sets of hyperparameter while tracking metrics specific to a particular problem or procedure. Once certain performance metrics are satisfied, the solutions may be deployed either on the cloud (e.g., a medical data processing platform) and/or on one or more physical devices (e.g., one or more surgical tools or medical instruments).

Data Acquisition

In tightly regulated fields such as healthcare, medical data acquisition may require special attention and can present specific challenges concerning patient privacy. One standard approach for acquiring medical data is to obtain RGB video data from a surgical procedure. While this seems like a straightforward approach, because of the risk of inadvertently exposing patient identifiers through the video stream, one must pay particular attention to removing sequences that can expose personal identifying information. The systems and methods of the present disclosure may be used to process the medical data, including surgical video data, to remove personal information and anonymize the medical data before it is used for model training and deployment.

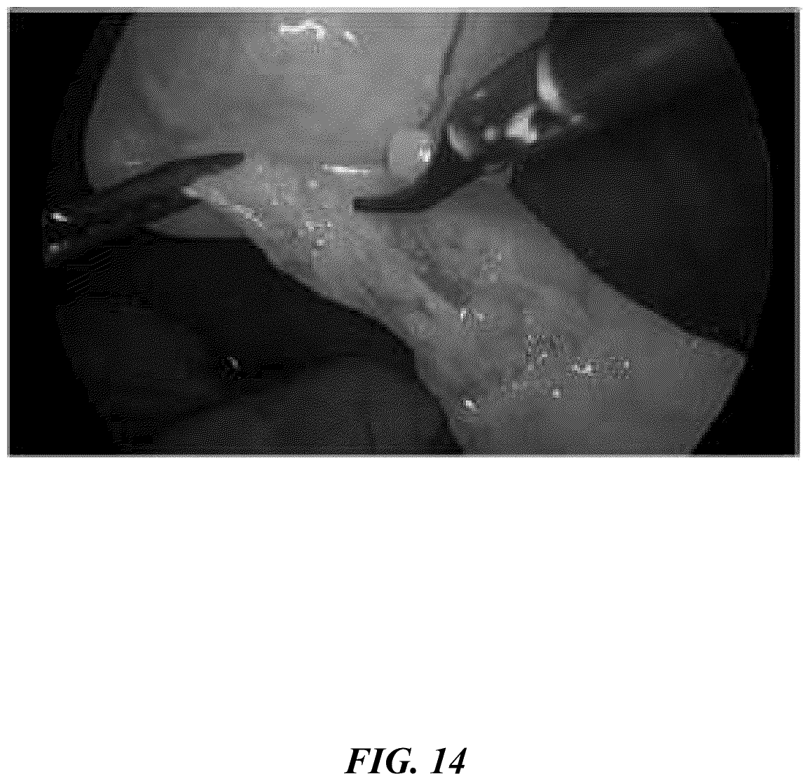

In some cases, the medical data (e.g., RGB images or videos of a surgical procedure) may be augmented or supplemented with additional information generated by the AI models, including for example, tool and tissue augmentation data. In some cases, the virtual surgical assistant may display such augmentations along with other types of medical data (e.g., as shown in FIG. 14) to a doctor or a surgeon in order to provide live surgical guidance or assistance and immediately benefit patient care. The augmented data may be displayed along with the RGB image or video data in real time as the data is being captured or obtained. In some cases, the augmented data may comprise, for example, one or more annotations as described elsewhere herein. In some cases, the augmented data may comprise one or more surgical or medical inferences generated based on the one or more annotations.

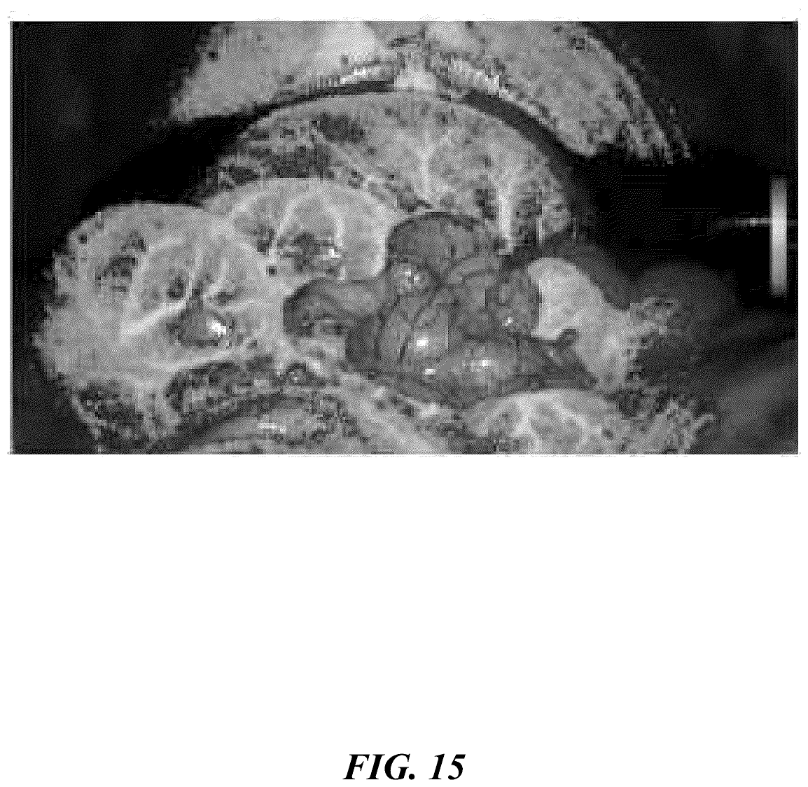

In some embodiments, the systems of the present disclosure may be used compatibly with various imaging platforms, including a vendor agnostic laparoscopic adapter that is configured to augment the RGB surgical video with real-time perfusion information without using any exogenous contrast agents. In some cases, the imaging platforms may comprise a hand-held imaging module with infrared capabilities, and a processing unit that allows recording of the infrared data to generate perfusion overlays that can be enabled on-demand by a surgeon. The platform may be based on any computer architecture and may use various graphics processing units for perfusion calculation and rendering. FIG. 15 shows an example of a perfusion overlay from the system with the un-perfused area shown in the center of the figure.

Data Annotation

Once the medical data is acquired and stripped of personal health information, the data may be annotated. In contrast to other domains such as autonomous vehicles, where anyone can recognize and annotate cars, crosswalks and road signs, the surgical data generally requires annotators with surgical expertise. While some objects, such as surgical tools, can be easily recognized by most people, specific anatomical structures and nuances specific to each patient requires surgical expert's annotations, which can be costly and time consuming. The systems and methods described above can be implemented to facilitate the annotation process and to compile annotations from various institutions and medical experts for model training.

Training

Once the medical data is collected, transformed into the correct format, and/or annotated, the medical data can be used to train one or more virtual surgical assistants. The training procedure may comprise an artificial intelligence (AI) development pipeline that is similar to the training procedures for machine learning (ML) models shown in FIG. 13. In some cases, each training session may be logged and versioned, including source code, hyper-parameters, and training datasets. This is particularly important in the healthcare field where a regulatory body might request this information, and where traceability is important. After training is completed and one or more desired metrics are achieved, the models may be deployed.

Deployment

When considering deployment, there is a regulatory component that needs to be considered in addition to the technical component. From a regulatory perspective, risk mitigation may influence the technical aspects of model deployment. While the virtual surgical assistants may not make any decisions during a surgical procedure, providing inaccurate information can still present a risk. It is important to identify possible failures scenarios and mitigation strategies to ensure the safety of the patient and medical staff. From a technical perspective, there are at least two types of deployment avenues: cloud deployment or edge deployment.

Cloud deployment may be implemented more easily, but may have some inherent limitations. In the case of virtual surgical assistants, real time inference is critical in the operating room and a cloud deployment may not always be feasible because of the overhead required by the data transfer. However, cloud deployment can still be used retrospectively on the recorded data to test future virtual assistants that are not yet ready for the operating room, or to allow surgeons to review the case and provide feedback. For real-time inferences, edge or device deployment may be the preferred approach. In such cases, a few aspects to consider include the architecture of the edge device and any possible power constraints. In the case of virtual surgical assistants, the power constraints are not necessarily a limitation, but should be considered, especially for edge cases. In some embodiments, multiple deployment options may be utilized. This may comprise a combination of cloud deployment and edge deployment.

Once a deployment architecture is selected, the next step is to get the model inference up and running. While using the training framework for deployment may seem like a logical step, performance may not be as expected, and the model may need to be further optimized for the specific architecture.

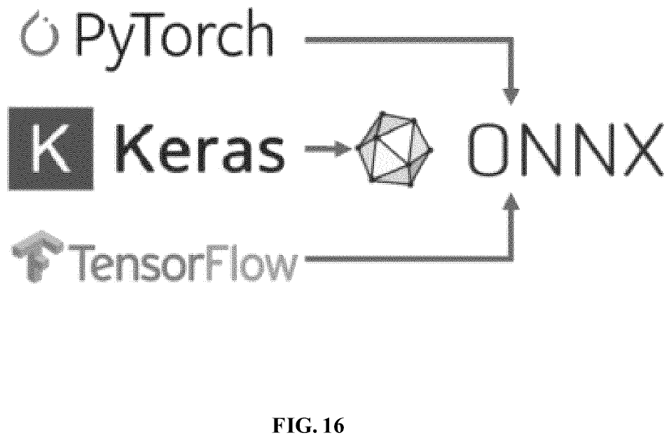

As shown in FIG. 16, the deployment pipeline may involve converting the model from one or more training frameworks such as PyTorch or TensorFlow to an open standard such as Open Neural Network Exchange (ONNX). Generally, this is an easy task and in PyTorch for example, only requires a single line of code. The call can create a representation of the model in a common file format using common sets of operators. In this format, the model can be tested on different hardware and software platforms using ONNX Runtime.

ONNX Runtime is a cross-platform inferencing and training accelerator that supports integration with various hardware acceleration libraries through an extensible framework called Execution Providers. ONNX currently supports about a dozen execution providers including the Compute Unified Device Architecture (CUDA) parallel computing platform and TensorRT high-performance deep learning inference SDK from Nvidia and the Microsoft DirectML low level application programming interface (API) for machine learning. ONNX Runtime can be used to easily run models on different types of hardware and operating systems by providing APIs for different programming languages including C, C#, Java, or Python. ONNX Runtime can be utilized for the real world deployment of virtual surgical assistants, both for cloud deployment and edge deployment.

Once the model is converted to ONNX, running inferences using the ONNX runtime is a simple task. For example, a user can quickly select an execution provider and run an inference session. One advantage of this approach is that a user can specify a list of execution providers, and any unsupported operations on a certain provider will be executed on the next provider specified. For example, a providers list of TensorRT followed by CUDA and CPU, will try to execute all the operations on TensorRT. If the operation is unsupported, the session will try CUDA before falling back to CPU execution.

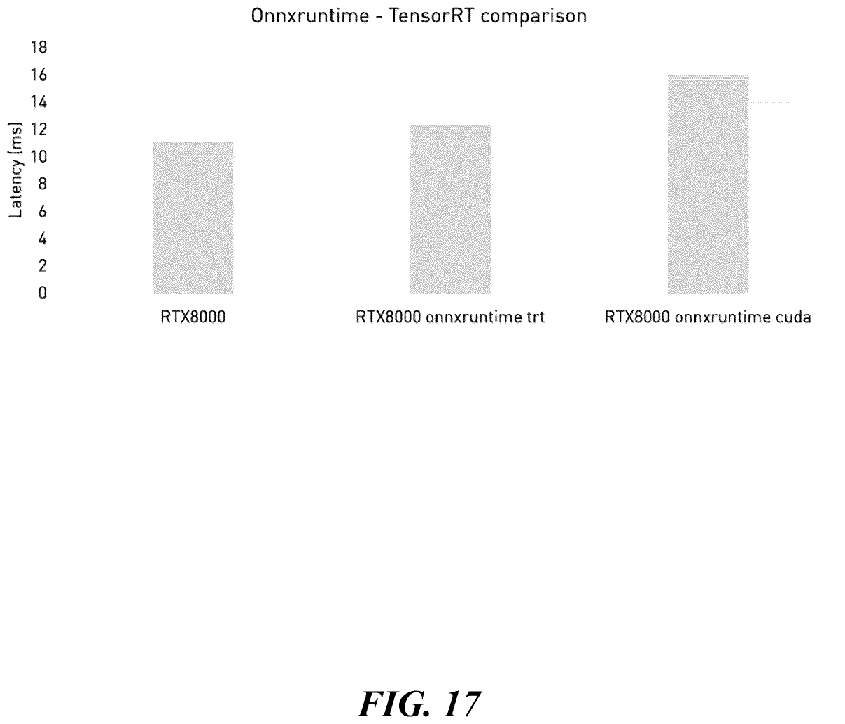

FIG. 17 shows the inference latencies between various ONNX runtime execution providers for a variant of the InceptionV3 convolutional neural network running on an Nvidia RTX8000 GPU with a batch size of 8 (i.e., 8 video frames). One can notice an improvement of about 20% when comparing the CUDA execution provider to the TensorRT execution provider. For reference, the left most bar shows the latency for a native TensorRT engine. This indicates that there is some overhead in the ONNX Runtime compared to the native TensorRT engine. However, the ease of implementation makes ONNX Runtime an ideal candidate for cloud deployment and depending on the situation even a good edge deployment solution. However, if this approach is not sufficient for a particular need or use case, the model may need to be converted using an optimize inference SDK such as TensorRT for Nvidia GPUs or SNPE (Snapdragon Neural Processing Engine) for Qualcomm hardware.

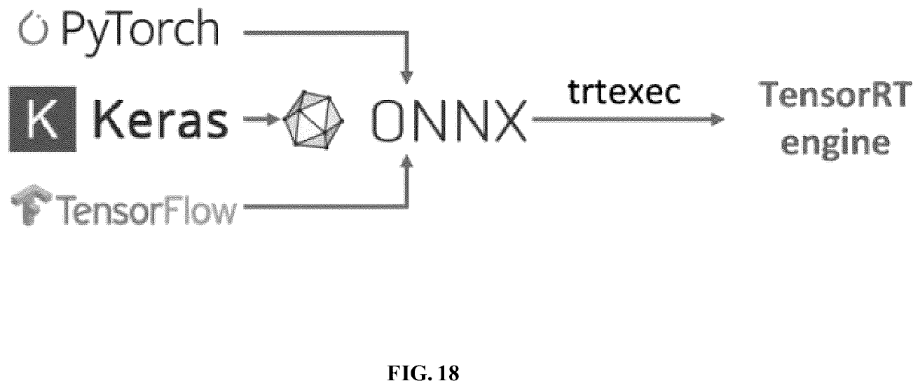

As shown in FIG. 18, the quickest path to creating a TensorRT engine is by taking the ONNX model created previously and using the trtexec command (a command line wrapper tool to quickly utilize TensorRT without having to develop a separate application). The trtexec command is useful for benchmarking networks on random data and for generating serialized engines from models. It does not require coding and besides generating the serialized engine, the command can be used to quickly benchmark models. Generating an engine requires a simple command that can also provide a lot of information about the model, including latencies and supported operations. Depending on the model, the results of the trtexec command can vary. In the best-case scenario, all the operations will be supported by the TensorRT SDK and the acceleration will be maximal. The command will also provide detailed latency metrics for the model. Additionally, if the hardware supports deep learning accelerators (DLAs), some operations might be supported by the accelerator as well. This will allow offloading of some operations from the GPU to the DLA and can provide more power efficient inferences. The generated serialized engine file can also be used during application development.

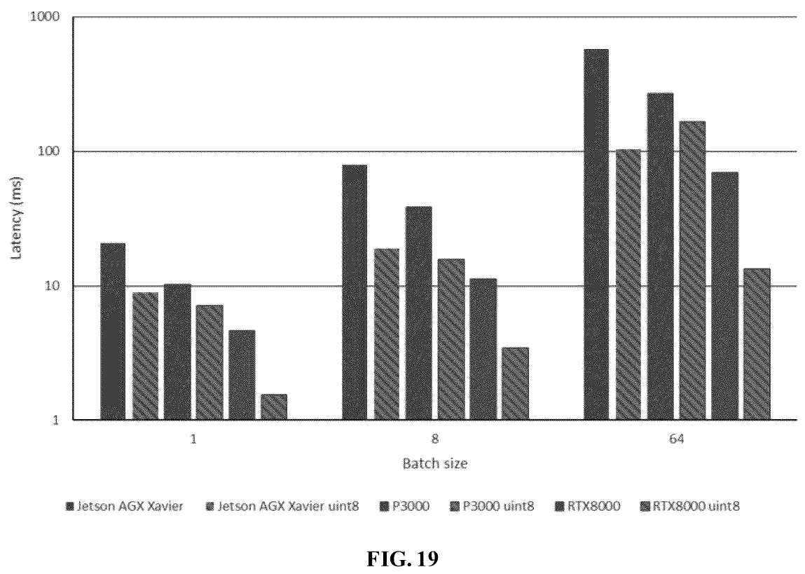

FIG. 19 shows a comparison of latencies across different devices including current generation hardware based on pascal architecture, an RTX8000 GPU, and the Jetson AGX Xavier. As expected, the RTX8000 had the best performance. When comparing current generation system powered by the Nvidia Quadro P3000 GPU against the Jetson AGX Xavier, the results were similar with a slight edge for the P3000 GPU. However, if the power budget is a concern, the Jetson AGX Xavier is the better solution. Additional acceleration can be achieved using int8 quantization at the cost of lower accuracy, but additional steps are required to create the calibration files specific to the dataset and might not always be feasible. As a compromise, 16-bit floating point inferences might be used if supported by the GPU architecture.

In some cases, depending on the model, some operations might not be supported by the TensorRT SDK. In such cases, there are a couple of alternatives. An optimal solution may depend on how far along one is in the development cycle and how strict the requirements are for the model. One can choose to write one or more TensorRT plugins for unsupported operations. Alternatively, one can modify the model to ensure that all the operation are supported out of the box, but this might not be the most time and cost-effective option considering the model training time.

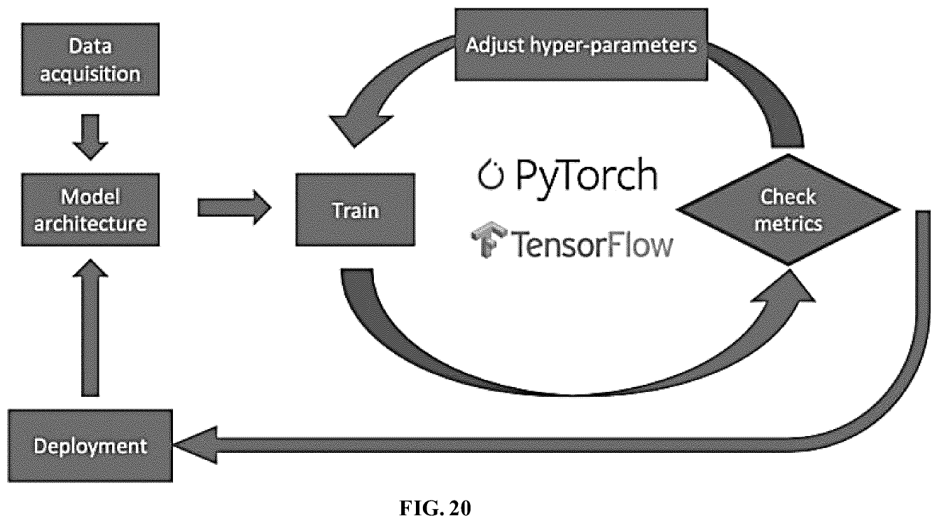

With these issues in mind, one may consider a more comprehensive training pipeline (e.g., as shown in FIG. 20) where the development architecture is used as an input when designing the models. While this might provide less flexibility during model development, minimizing the number of custom operations required for development may be beneficial in the long run. Developing the models with the deployment hardware in mind may allow for latency testing, operational support testing, and/or memory usage testing before additional time is invested in model training. Moreover, this process can be used to determine if the current hardware is underpowered, thereby allowing for early adjustments to hardware and/or software during model development.

When deploying virtual surgical assistants in the operating room, it is important to always start with the deployment architecture in mind and to design models for the specific deployment architecture. It is also important to determine early on if custom operations are critical, and to weigh the cost and benefits of using them. Further, it is important to use tools such as ONNX Runtime to quickly test models across operating systems and hardware architectures, and only optimize in the final stages if lower latencies are required. From the hardware perspective, it is important to also consider non-AI tasks that require GPU usage and to pick a computing or processing device with enough overhead to support additional features.

In another aspect, the present disclosure provides computer systems that are programmed or otherwise configured to implement methods of the disclosure. Referring back to FIG. 11, the computer system 2001 may be programmed or otherwise configured to implement a method for deploying one or more models. The computer system 2001 may be configured to, for example, acquire medical or surgical data, train a model based on the medical or surgical data, evaluate one or more performance metrics for the model, adjust the model by changing or modifying one or more hyperparameters, and deploy the trained model. The computer system 2001 can be an electronic device of a user or a computer system that is remotely located with respect to the electronic device. The electronic device can be a mobile electronic device.

The computer system 2001 may include a central processing unit (CPU, also “processor” and “computer processor” herein) 2005, which can be a single core or multi core processor, or a plurality of processors for parallel processing. The computer system 2001 also includes memory or memory location 2010 (e.g., random-access memory, read-only memory, flash memory), electronic storage unit 2015 (e.g., hard disk), communication interface 2020 (e.g., network adapter) for communicating with one or more other systems, and peripheral devices 2025, such as cache, other memory, data storage and/or electronic display adapters. The memory 2010, storage unit 2015, interface 2020 and peripheral devices 2025 are in communication with the CPU 2005 through a communication bus (solid lines), such as a motherboard. The storage unit 2015 can be a data storage unit (or data repository) for storing data. The computer system 2001 can be operatively coupled to a computer network (“network”) 2030 with the aid of the communication interface 2020. The network 2030 can be the Internet, an internet and/or extranet, or an intranet and/or extranet that is in communication with the Internet. The network 2030 in some cases is a telecommunication and/or data network. The network 2030 can include one or more computer servers, which can enable distributed computing, such as cloud computing. The network 2030, in some cases with the aid of the computer system 2001, can implement a peer-to-peer network, which may enable devices coupled to the computer system 2001 to behave as a client or a server.

The CPU 2005 can execute a sequence of machine-readable instructions, which can be embodied in a program or software. The instructions may be stored in a memory location, such as the memory 2010. The instructions can be directed to the CPU 2005, which can subsequently program or otherwise configure the CPU 2005 to implement methods of the present disclosure. Examples of operations performed by the CPU 2005 can include fetch, decode, execute, and writeback.

The CPU 2005 can be part of a circuit, such as an integrated circuit. One or more other components of the system 2001 can be included in the circuit. In some cases, the circuit is an application specific integrated circuit (ASIC).

The storage unit 2015 can store files, such as drivers, libraries and saved programs. The storage unit 2015 can store user data, e.g., user preferences and user programs. The computer system 2001 in some cases can include one or more additional data storage units that are located external to the computer system 2001 (e.g., on a remote server that is in communication with the computer system 2001 through an intranet or the Internet).

The computer system 2001 can communicate with one or more remote computer systems through the network 2030. For instance, the computer system 2001 can communicate with a remote computer system of a user (e.g., a doctor, a surgeon, an operator, a healthcare provider, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2001 via the network 2030.

Methods as described herein can be implemented by way of machine (e.g., computer processor) executable code stored on an electronic storage location of the computer system 2001, such as, for example, on the memory 2010 or electronic storage unit 2015. The machine executable or machine readable code can be provided in the form of software. During use, the code can be executed by the processor 2005. In some cases, the code can be retrieved from the storage unit 2015 and stored on the memory 2010 for ready access by the processor 2005. In some situations, the electronic storage unit 2015 can be precluded, and machine-executable instructions are stored on memory 2010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 2001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2001 can include or be in communication with an electronic display 2035 that comprises a user interface (UI) 2040 for providing, for example, a portal for a doctor or a surgeon to view one or more medical inferences associated with a live procedure. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2005. For example, the algorithm may be configured to acquire medical or surgical data, train a model based on the medical or surgical data, evaluate one or more performance metrics for the model, adjust the model by changing or modifying one or more hyperparameters, and deploy the trained model. In any of the embodiments described herein, one or more graphics processing units (GPUs) or deep learning accelerators (DLAs) may be used to implement the systems and methods of the present disclosure.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-169. (canceled)

170. A method for generating medical insights, comprising:
(a) obtaining medical data associated with a surgical procedure using one or more medical tools or instruments;
(b) processing the medical data using, wherein the one or more medical algorithms or models are deployed or implemented on or by (i) the one or more medical tools or instruments or (ii) a data processing platform;
(c) generating one or more insights or inferences based on the processed medical data; and
(d) providing the one or more insights one or more medical algorithms or models or inferences for the surgical procedure to at least one of (i) a device in an operating room and (ii) a user via the data processing platform.

171. The method of claim 170, further comprising registering the one or more medical tools or instruments with the data processing platform, and/or uploading the medical data or the processed medical data from the one or more medical tools or instruments to the data processing platform.

172. (canceled)

173. The method of claim 170, wherein the one or more medical algorithms or models are trained with one or more medical data sets.

174. The method of claim 173, wherein the one or more medical data sets are associated with one or more reference surgical procedures of a same or similar type as the surgical procedure.

175. The method of claim 170, wherein the one or more medical tools or instruments comprise an imaging device.

176. The method of claim 175, wherein the imaging device is configured for RGB imaging, laser speckle imaging, fluorescence imaging, or time of flight imaging.

177. The method of claim 170, wherein the medical data comprises one or more images or videos of the surgical procedure or one or more steps of the surgical procedure.

178. The method of claim 170, wherein processing the medical data comprises determining or classifying one or more features, patterns, or attributes of the medical data.

179. The method of claim 170, wherein the one or more insights comprise tool identification, tool tracking, surgical phase timeline, critical view detection, tissue structure segmentation, and/or feature detection.

180. The method of claim 170, wherein the one or more medical algorithms or models are configured to perform one or more of the following: tissue tracking, augmentation of medical data with depth information, tool segmentation, phase of surgery breakdown, critical view detection, tissue structure segmentation, feature detection, deidentification or anonymization of the medical data.

181. (canceled)

182. (canceled)

183. (canceled)

184. The method of claim 170, wherein the one or more medical algorithms or models are configured to provide live guidance based on a detection of one or more tools, surgical phases, critical views, or one or more biological, anatomical, physiological, or morphological features in or near the surgical scene.

185. The method of claim 170, wherein the one or more medical algorithms or models are configured to generate synthetic data for simulation and/or extrapolation.

186. The method of claim 170, wherein the one or more medical algorithms or models are configured to assess a quality of the medical data.

187. The method of claim 170, wherein the one or more medical algorithms or models are configured to generate an overlay comprising (i) one or more RGB images or videos of the surgical scene and (ii) one or more additional images or videos of the surgical procedure, wherein the one or more additional images or videos comprise fluorescence data, laser speckle data, perfusion data, depth information, or internal body structure or tissue information.

188. The method of claim 170, wherein the one or more medical algorithms or models are configured to provide one or more surgical inferences.

189. The method of claim 188, wherein the one or more inferences comprise a determination of whether a tissue is alive.

190. The method of claim 188, wherein the one or more inferences comprise a determination of where to make a cut or an incision.

191. The method of claim 170, wherein the one or more medical algorithms or models are configured to provide virtual surgical assistance to a surgeon or a doctor performing the surgical procedure.

192. The method of claim 170, wherein the one or more medical algorithms or models are trained using one or more algorithms configured to implement exponential smoothing, single exponential smoothing, double exponential smoothing, triple exponential smoothing, Holt-Winters exponential smoothing, autoregressions, moving averages, autoregressive moving averages, autoregressive integrated moving averages, seasonal autoregressive integrated moving averages, vector autoregressions, or vector autoregression moving averages.

193. The method of claim 170, wherein the one or more medical algorithms or models are trained using deep learning, reinforcement learning, transfer learning, image thresholding, color-based image segmentation, regression analysis, support vector machines, one or more decision trees or random forests associated with the one or more decision trees.

194. The method of claim 170, wherein the one or more medical algorithms or models are trained using data augmentation techniques or generative adversarial networks.
